(19) United States
(12) Patent Application Publication
(10) Pub. No.: US 2025/0107889 A1
(43) Pub. Date: Apr. 3, 2025

(54) PROSTHETIC VALVES FOR IMPLANTATION IN CALCIFIED NATIVE VALVES

(71) Applicant: EDWARDS LIFESCIENCES CORPORATION, Irvine, CA (US)

(72) Inventors: Kevin M. Stewart, Powell, TN (US); Siddharth Vad, Irvine, CA (US); Jesse Robert Edwards, Silverado, CA (US); David Robert Landon, Huntington Beach, CA (US); Jonathan Yeh, Brea, CA (US); Hieu Minh Luong, Westminster, CA (US); Harish Manickam Srinimukesh, Costa Mesa, CA (US); Alyssa Nicole Knauer, Newport Beach, CA (US)

(21) Appl. No.: 18/981,502

(22) Filed: Dec. 14, 2024

Related U.S. Application Data
(63) Continuation of application No. PCT/US2023/026030, filed on Jun. 23, 2023.
(60) Provisional application No. 63/355,503, filed on Jun. 24, 2022.

(51) Int. Cl. A61F 2/24; A61L 27/36

(52) U.S. Cl. CPC A61F 2/2418; A61F 2/2478; A61L 27/3604; A61F 2210/0014; A61F 2220/0008; A61F 2230/0069; A61F 2250/0003; A61L 2430/20

(57) ABSTRACT

Apparatuses, systems, and methods for prosthetic valves. An implantation site may comprise a native heart valve or another implantation site in examples. Examples may be utilized for improved anchoring and sealing of flow (e.g., paravalvular leakage) with a native heart valve having calcification. Examples may reduce the possibility of obstruction of a left ventricular outflow tract (LVOT) of a heart, whether resulting from implantation of a prosthetic valve or otherwise.

### CROSS REFERENCE TO RELATED APPLICATIONS

This application is a continuation of International Application No. PCT/US2023/026030, filed Jun. 23, 2023, which designates the United States and was published in English by the International Bureau on Dec. 28, 2023 as WO2023/250114, which claims the benefit of U.S. Provisional Application No. 63/355,503, filed Jun. 24, 2022, the entire contents of each of which are hereby incorporated by reference.

### BACKGROUND

### Field

The present disclosure primarily relates to prosthetic heart valves, and delivery systems for implanting heart valves.

### Background

Human heart valves, which include the aortic, pulmonary, mitral, and tricuspid valves, function essentially as one-way valves operating in synchronization with the pumping heart. The valves allow blood to flow downstream, but block blood from flowing upstream. Diseased heart valves exhibit impairments such as narrowing of the valve or regurgitation, which inhibit the valves' ability to control blood flow. Such impairments reduce the heart's blood-pumping efficiency and can be a debilitating and life-threatening condition. For example, valve insufficiency can lead to conditions such as heart hypertrophy and dilation of the ventricle. Thus, extensive efforts have been made to develop methods and apparatuses to repair or replace defective heart valves.

Prostheses exist to correct problems associated with defective native heart valves. For example, mechanical and tissue-based heart valve prostheses can be used to replace defective native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and, more recently, through non-invasive percutaneous procedures.

These replacement valves are often intended to allow fluid flow therethrough, while sealing or blocking fluid flow on the outside of the replacement valve (paravalvular leakage (PVL)). Effective sealing can be especially challenging if the native heart valve (e.g., annulus) has a distorted shape or has calcification, which may create an irregular surface. A distorted and/or calcified native heart valve may also reduce the possibility of proper anchoring of the replacement valve within the native heart valve.

### SUMMARY

Examples of prosthetic valves may provide improved anchoring. Examples of prosthetic valves may also reduce paravalvular leakage by enhancing the seal around the exterior of the prosthetic valve. Prosthetic valves of this type are particularly useful for replacing a calcified native heart valve, such as a calcified mitral or tricuspid valve. Examples may reduce the possibility of obstruction of a left ventricular outflow tract (LVOT) of a heart, whether resulting from implantation of a prosthetic valve or otherwise. Various other improvements are disclosed.

Examples disclosed herein may include a prosthetic valve configured to be deployed within a native valve. The prosthetic valve may include one or more prosthetic valve leaflets positioned in a flow channel and a valve body configured to support the one or more prosthetic valve leaflets. The valve body may include a proximal anchor comprising a first flange configured to extend radially outward from the flow channel, and a distal anchor comprising a second flange configured to extend radially outward from the flow channel.

Examples disclosed herein may include a prosthetic valve configured to be deployed within a native valve. The prosthetic valve may include an inner support stent having an inlet end portion and an outlet end portion, the inner support stent made from a shape memory material. The prosthetic valve may include a valve portion positioned within a passageway of the inner support stent, wherein the valve portion comprises a plurality of leaflets made from pericardium, wherein the valve portion permits flow of blood through the passageway in one direction for replacing the function of the native valve. The prosthetic valve may include an outer conformable structure surrounding the inner support stent. The outer conformable structure may be adapted to conform to calcification on the native valve for enhancing sealing and anchoring to the native valve.

Examples disclosed herein may include methods of implanting a prosthetic valve within a native valve. The prosthetic valve may comprise one or more prosthetic valve leaflets configured to be positioned in a flow channel, and a valve body configured to support the one or more prosthetic valve leaflets, the valve body including: a proximal anchor comprising a first flange configured to extend radially outward from the flow channel, and a distal anchor comprising a second flange configured to extend radially outward from the flow channel.

Examples disclosed herein may include a prosthetic valve configured to be deployed to a native valve. The prosthetic valve may include one or more prosthetic valve leaflets configured to be positioned in a flow channel; and a valve body configured to support the one or more prosthetic valve leaflets, the valve body including an atrial anchor forming a ring about the flow channel.

Examples disclosed herein may include a prosthetic valve for replacing a native valve. The prosthetic valve may include a support structure for deployment on an upstream side of the native valve and including an atrial anchor forming a ring about a flow channel. A plurality of leaflets made from pericardium may be coupled to the support structure and disposed within a passageway of the support structure for providing one-way flow of blood.

Examples disclosed herein may include a method of implanting a prosthetic valve within a native valve. The prosthetic valve may comprise one or more prosthetic valve leaflets configured to be positioned in a flow channel, and a valve body configured to support the one or more prosthetic valve leaflets, the valve body including an atrial anchor forming a ring about the flow channel.

The prosthetic valve system may comprise one or more prosthetic valve leaflets configured to be positioned in a flow channel; a valve body supporting the one or more prosthetic valve leaflets and surrounding the flow channel; and a dock comprising an elongate body having a first end portion and a second end portion and configured to form a crescent shape, the first end portion including a first penetrating body configured to anchor to heart tissue and the second end portion including a second penetrating body configured to anchor to heart tissue, the dock configured to dock with the valve body.

Examples disclosed herein may include a method comprising deploying a dock proximate a native valve, the dock comprising an elongate body having a first end portion and a second end portion and configured to form a crescent shape, the first end portion including a first penetrating body configured to anchor to heart tissue and the second end portion including a second penetrating body configured to anchor to heart tissue; and deploying a valve body supporting one or more prosthetic valve leaflets to the dock.

In another example, a prosthetic valve may comprise one or more prosthetic valve leaflets configured to be positioned in a flow channel; and a valve body configured to support the one or more prosthetic valve leaflets, the valve body including an outer perimeter including a first portion forming at least a quarter of the outer perimeter and a second portion forming a remainder of the outer perimeter, and the valve body includes one or more distal anchors protruding from the second portion and lacks distal anchors protruding from the first portion.

In another example, a prosthetic valve for implantation within a native valve may comprise a support structure having an outer perimeter including a first portion forming at least a quarter of the outer perimeter and a second portion forming a remainder of the outer perimeter, and the support structure includes one or more distal anchors for capturing a native valve leaflet protruding from the second portion and lacks distal anchors protruding from the first portion. The prosthetic valve may include one or more prosthetic valve leaflets for being positioned in a flow channel of the support structure.

In another example, a prosthetic valve may comprise one or more prosthetic valve leaflets configured to be positioned in a flow channel; and a valve body configured to support the one or more prosthetic valve leaflets and including a sealing body having a first portion and a second portion, the first portion impeding fluid flow at a height of the valve body and the second portion being at the height of the first portion and being at a circumferentially offset position from the position of the first portion, the second portion being recessed to allow for fluid flow through at the circumferentially offset position.

In another example, a prosthetic valve may comprise one or more prosthetic valve leaflets configured to be positioned in a flow channel, and a valve body configured to support the one or more prosthetic valve leaflets and including a sealing body having a first portion and a second portion, the first portion impeding fluid flow at a height of the valve body and the second portion being at the height of the first portion and being at a circumferentially offset position from the position of the first portion, the second portion being recessed to allow for fluid flow through at the circumferentially offset position.

In another example, a prosthetic valve may comprise one or more prosthetic valve leaflets configured to be positioned in a flow channel; a valve body configured to support the one or more prosthetic valve leaflets; and at least one distal anchor coupled to the valve body, the at least one distal anchor including a hinge forming a loop and a straight portion configured to extend radially outward from the loop.

Other examples disclosed herein may include a compression system for a heart. The system may comprise a first compressive body configured to be positioned on a first side of an interventricular septum of the heart proximate a left ventricular outflow tract; a second compressive body configured to be positioned on a second side of the interventricular septum or on a free wall of a right ventricle of the heart; and a tether configured to compress the first compressive body and the second compressive body together to increase a size of the left ventricular outflow tract.

Other examples disclosed herein may include a compression system for a heart. The compression system may comprise a first compressive body for being positioned on a first side of an interventricular septum of the heart proximate a left ventricular outflow tract. The compression system may comprise second compressive body for being positioned on a second side of the interventricular septum or on a free wall of a right ventricle of the heart. The compression system may comprise a tether for decreasing a distance between the first compressive body and the second compressive body for improving flow through the left ventricular outflow tract.

Examples disclosed herein may include a system for a heart. The system may comprise a stent configured to be deployed into the heart proximate a left ventricular outflow tract of the heart and including a flow channel for fluid to pass through the left ventricular outflow tract.

Examples disclosed herein may include a system for a heart. The system may comprise a first prosthetic heart valve configured to be implanted in an aortic valve of the heart; and a second prosthetic heart valve coupled to the first prosthetic heart valve and configured to be implanted in a mitral valve of the heart.

In another example, a prosthetic valve may comprise one or more prosthetic valve leaflets configured to be positioned in a flow channel; a valve body configured to support the one or more prosthetic valve leaflets; and at least one distal arm coupled to the valve body and configured to apply a force to an interventricular septum between a left ventricle and a right ventricle of the heart.

### BRIEF DESCRIPTION OF THE DRAWINGS

Features and advantages of the systems, apparatuses, and methods as disclosed herein will become appreciated as the same become better understood with reference to the specification, claims, and appended drawings wherein:

FIG. 1A illustrates an upper perspective view of a prosthetic valve according to examples of the present disclosure.

FIG. 1B illustrates a bottom perspective view of the prosthetic valve shown in FIG. 1A.

FIG. 2 illustrates a side cross sectional schematic view of the prosthetic valve shown in FIG. 1A.

FIG. 3 illustrates a schematic view of a delivery apparatus approaching an implantation site.

FIG. 4 illustrates a side cross sectional schematic view of the prosthetic valve shown in FIG. 1A deployed to a native heart valve.

FIG. 5 illustrates a side cross sectional schematic view of a native heart valve having calcification.

FIG. 6A illustrates a side cross sectional view of an anchor.

FIG. 6B illustrates a side cross sectional view of the anchor shown in FIG. 6A in an extended position.

FIG. 6C illustrates a side cross sectional view of the anchor shown in FIG. 6B in a position that is rotated from the position shown in FIG. 6B.

FIG. 7A illustrates a side cross sectional schematic view of a prosthetic valve approaching a native heart valve.

FIG. 7B illustrates a side cross sectional schematic view of the prosthetic valve shown in FIG. 7A deployed to a native heart valve.

FIG. 8A illustrates a perspective view of a prosthetic valve.

FIG. 8B illustrates a side cross sectional schematic view of the prosthetic valve shown in FIG. 8A deployed to a native heart valve.

FIG. 9A illustrates a side cross sectional schematic view of a prosthetic valve approaching a native heart valve.

FIG. 9B illustrates a side cross sectional schematic view of the prosthetic valve shown in FIG. 9A deployed to a native heart valve.

FIG. 10 illustrates a side cross sectional schematic view of a prosthetic valve deployed to a native heart valve.

FIG. 11A illustrates a flat pattern of a prosthetic valve for deployment to a native heart valve.

FIG. 11B illustrates a perspective view of a prosthetic valve utilizing the flat pattern shown in FIG. 11A.

FIG. 12 illustrates a side cross sectional view of a prosthetic valve deployed to a native heart valve.

FIG. 13 illustrates a side cross sectional view of a prosthetic valve deployed to a native heart valve.

FIG. 14A illustrates a side cross sectional view of a prosthetic valve deployed to a native heart valve.

FIG. 14B illustrates a top view of the prosthetic valve shown in FIG. 14A deployed to a native heart valve.

FIG. 15 illustrates a side cross sectional view of a prosthetic valve deployed to a native heart valve.

FIG. 16A illustrates a perspective view of a prosthetic valve and a plurality of anchors.

FIG. 16B illustrates a side cross sectional view of the prosthetic valve shown in FIG. 16A deployed to a native heart valve.

FIG. 17A illustrates a perspective view of a dock for a prosthetic valve.

FIG. 17B illustrates a perspective view of a prosthetic valve.

FIG. 17C illustrates a side schematic view of the dock shown in FIG. 17A deployed to a native heart valve.

FIG. 17D illustrates a side schematic view of a prosthetic valve shown in FIG. 17B deployed to a dock as shown in FIG. 17C.

FIG. 18A illustrates a perspective view of a prosthetic valve.

FIG. 18B illustrates a top view of the prosthetic valve shown in FIG. 18A.

FIG. 18C illustrates a perspective view of a prosthetic valve.

FIG. 18D illustrates a top view of the prosthetic valve shown in FIG. 18C.

FIG. 19A illustrates a perspective view of a prosthetic valve.

FIG. 19B illustrates a side schematic view of the prosthetic valve shown in FIG. 19A deployed to a native heart valve.

FIG. 20A illustrates a side schematic view of a left ventricle.

FIG. 20B illustrates a perspective view of an implant for applying a force to an interventricular septum.

FIG. 20C illustrates a side schematic view of the implant shown in FIG. 20B applied to an interventricular septum.

FIG. 20D illustrates a side schematic view of a prosthetic heart valve applied to a native heart valve.

FIG. 21 illustrates a side schematic view of an implant applied to an interventricular septum.

FIG. 22 illustrates a side schematic view of a stent deployed proximate a left ventricular outflow tract.

FIG. 23 illustrates a side schematic view of a stent deployed proximate a left ventricular outflow tract.

FIG. 24 illustrates a side schematic view of a stent deployed proximate a left ventricular outflow tract.

FIG. 25A illustrates a side schematic view of a stent deployed proximate a left ventricular outflow tract and coupled to a prosthetic valve.

FIG. 25B illustrates a side schematic view of a stent deployed proximate a left ventricular outflow tract and coupled to a prosthetic valve.

FIG. 26 illustrates a side schematic view of a stent deployed proximate a left ventricular outflow tract and coupled to a prosthetic valve.

FIG. 27 illustrates a side schematic view of a prosthetic valve deployed to a mitral valve and coupled to a prosthetic valve deployed to an aortic valve.

FIG. 28A illustrates a perspective view of a prosthetic valve.

FIG. 28B illustrates a side view of the prosthetic valve shown in FIG. 28A.

### DETAILED DESCRIPTION

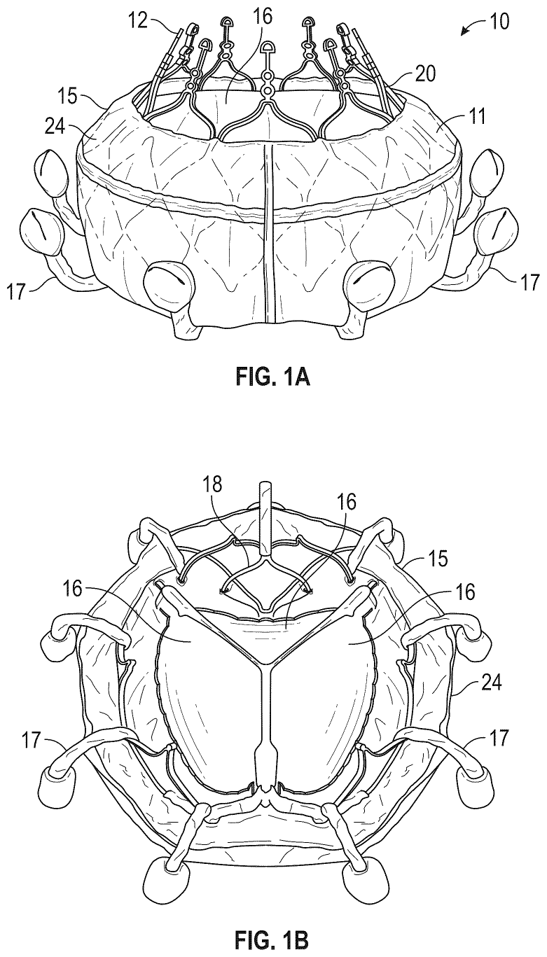

FIG. 1 illustrates a perspective view of a prosthetic valve 10 in the form of a replacement heart valve. The prosthetic valve 10 may be configured to be deployed within a portion of a patient's body. The prosthetic valve 10, for example, may be deployed to an annulus of a native valve, which may comprise a native mitral valve or a native tricuspid valve. In examples, other implantation locations may be utilized such as within an aortic or pulmonary valve, or in other valves or locations within a patient's body as desired.

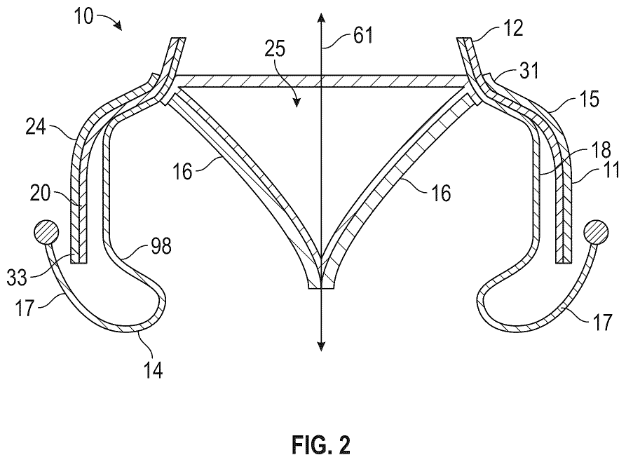

The prosthetic valve 10 may include a proximal end 12 (or inlet end portion) and a distal end 14 (or outlet end portion) (marked in FIG. 2), and a length therebetween. The prosthetic valve 10 may further include a valve portion including one or more prosthetic valve leaflets 16, or a plurality of prosthetic valve leaflets 16, configured to be positioned in a flow channel for controlling flow through the valve 10. The leaflets 16 may be made from pericardium or another material as desired. The prosthetic valve leaflets 16 may be configured to move between opened and closed states to mimic and replace the operation of native valve leaflets. The valve portion may permit flow of blood through the flow channel or passageway in one direction for replacing the function of the native valve.

In examples, the prosthetic valve leaflets 16 may be coupled to a valve body or support structure 15 that may be configured to support the one or more prosthetic valve leaflets 16. The support structure 15 may include an inner support stent having a frame (e.g., a valve frame or inner frame 18 and an outer support stent having an outer frame 20, among other forms of frames) and a sealing body 11. A valve frame or inner frame 18 is shown in FIG. 1B and in the cross sectional view of FIG. 2. An outer frame 20 is shown in FIGS. 1A and 2. The outer frame 20 may be part of the sealing body 11 and may be spaced from the inner frame 18. The support structure 15 may be made of a shape memory material such as Nitinol or another form of shape memory material in examples.

Referring to FIGS. 1A and 2, the prosthetic valve 10 may include one or more anchors 17 that may be coupled to the prosthetic valve leaflets 16. The anchors 17 may each be configured to anchor the prosthetic valve leaflets 16 to a portion of a patient's heart, which may comprise a native valve. The anchors 17 may particularly be configured to anchor to the native valve leaflets of the patient's heart. The anchors 17 may extend around the native valve leaflets to anchor to the native valve leaflets. The anchors 17 may comprise distal anchors positioned at the distal end 14 of the valve 10, or in examples may be positioned in another position as desired.

Each anchor 17 may be configured as a protruding arm configured to extend distally and then curve in a proximal direction to the tip of the respective one of the anchors 17. Such a configuration may allow the anchor 17 to extend around a native leaflet and around the distal tip of the leaflet, to hook over the distal tip of the native valve leaflet and be positioned radially outward of an outward facing surface of a leaflet of the native valve. The anchors 17 may be configured to be in a hooked configuration as shown in FIGS. 1A-2 for example. The anchor 17 may thus resist a force applied in the atrial or proximal direction to the valve 10 and may anchor the valve 10 within the native valve annulus. Other configurations of anchors 17 may be utilized in examples as desired.

The prosthetic valve leaflets 16 may surround a passageway or flow channel 25 as marked in FIG. 2 and may move between open and closed states to control flow through the flow channel 25. As shown in FIG. 2, the proximal end of the prosthetic valve 10 may comprise an inflow end of the valve 10, and the distal end of the prosthetic valve 10 may comprise an outflow end, although other configurations may be utilized as desired. The prosthetic valve leaflets 16 may be positioned around a central axis 61 of the prosthetic valve 10. The inner frame 18 and outer frame 20 may each surround the central axis 61 of the prosthetic valve 10.

Referring back to FIG. 1, the prosthetic valve 10 may include an outer support stent having a sealing body 11. The sealing body 11 may be positioned radially outward from the prosthetic valve leaflets 16 and may be configured to seal against a portion of the native valve. The sealing body 11 may comprise the outer surface of the valve 10. The sealing body 11 may define the outer diameter of the valve 10 and may comprise the outer periphery of the valve 10. The sealing body 11 may include a proximal portion having a proximal end 31 and may include a distal portion having a distal end 33 (marked in FIG. 2).

Referring to the cross sectional view of FIG. 2, the sealing body 11 may include a frame 20 and a sealing skirt 24, or in examples may comprise only a frame or only a sealing skirt as desired. The frame 20 may comprise an outer frame that is positioned radially outward from the inner frame 18. The sealing skirt 24 may be coupled to the outer frame 20 and may comprise the outer portion of the sealing body 11 as shown in FIG. 1A.

The sealing skirt 24 may be made of a material that resists fluid flow therethrough, such as a cloth material, woven material, or other material such as a polymer or other material that resists fluid flow therethrough. The material may comprise a fabric. A variety of materials may be utilized for the skirt 24 as desired.

The sealing body 11 may be configured to abut a portion of the patient's heart to reduce fluid flow. The skirt 24 may be configured to seal a portion of the native valve annulus. For example, the sealing body 11 may abut a surface of a patient's native valve leaflet to reduce fluid flow between the sealing body 11 and the native leaflet. The sealing body 11 may be configured to abut other portions of the patient's heart to reduce fluid flow as desired.

In examples, the sealing body 11 may be flexible to allow for movement and conformability to a native valve annulus.

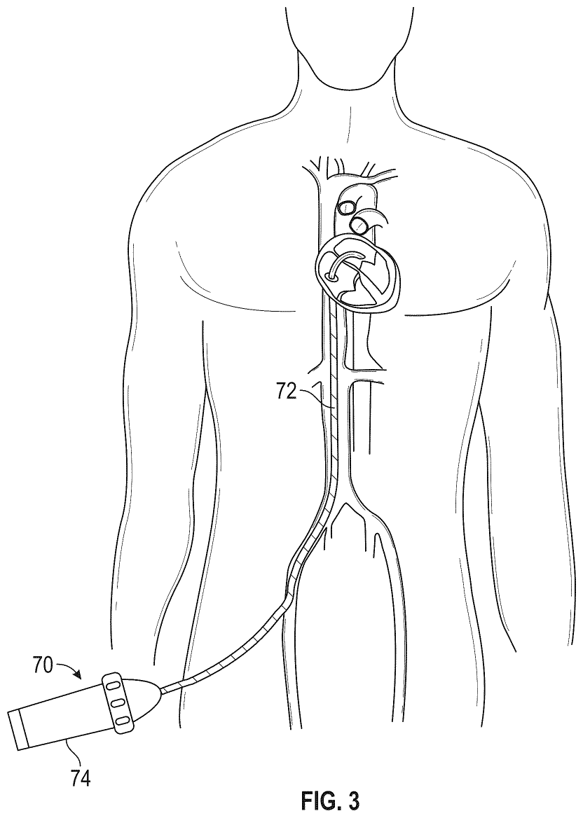

FIG. 3 illustrates advancement of a delivery system 70 for deployment of the prosthetic valve 10 to an implantation site. The delivery system 70 may include an elongate shaft 72 having a proximal portion and a distal portion, with the proximal portion coupled to a housing in the form of a handle 74. The delivery system 70 may be advanced through the vasculature of a patient, which may include the femoral vein as shown in FIG. 3. Other entries may be utilized in examples, including transapical, or via surgical methods such as thoracotomy or open heart surgery.

In examples, the prosthetic valve 10 may be positioned within an implant retention area of the delivery system 70 and may be covered with a capsule or may otherwise be retained prior to deployment. The prosthetic valve 10 may be deployed as a self-expanding prosthetic. The shape memory material of the support structure may allow the valve 10 to self-expand. In examples, however, the prosthetic valve may be a balloon-expandable prosthetic (e.g., positioned upon an inflatable body or balloon upon entry into the patient's body, or slid onto an inflatable body or balloon within the patient's body), or may be mechanically expanded, among other forms of deployment.

The delivery system 70 may be advanced to pass into an atrium of a heart and may pass transeptally into another atrium (e.g., from the right atrium to the left atrium) to reach an implantation site. Such a delivery approach may be utilized for mitral native valve access for example. In examples, the delivery system 70 may extend to the right atrium for tricuspid access, or other delivery approaches to other implantation sites may be utilized in examples as desired.

The prosthetic valve 10 may be held in a compressed configuration within a capsule of the delivery system 70. The anchors 17 may be advanced and may deploy radially outward from the capsule.

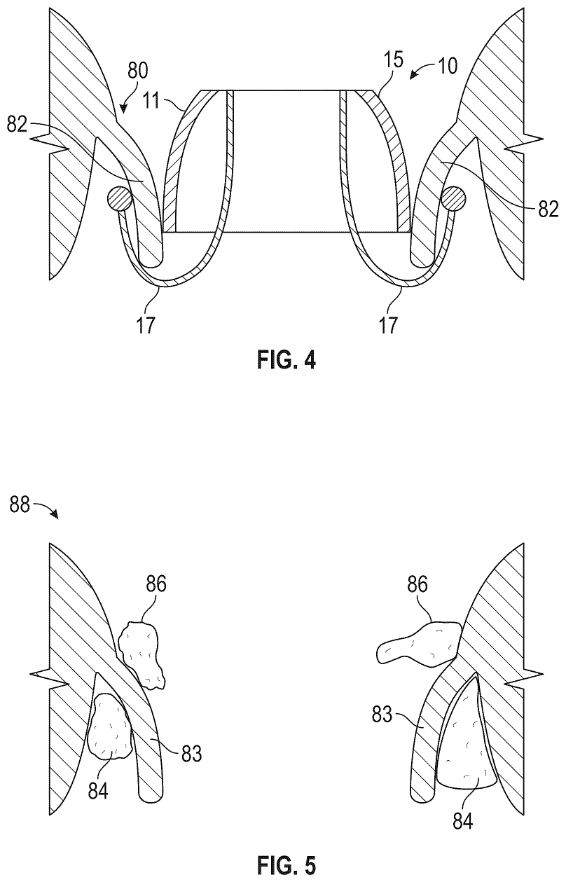

FIG. 4 illustrates the prosthetic valve 10 deployed to the native valve 80. The sealing body 11 may extend radially outward to contact the inward facing surfaces of the native valve leaflets 82. The anchors 17 may hook over native valve leaflets 82 such that the tips of the anchors 17 are positioned radially outward of the native valve leaflets 82.

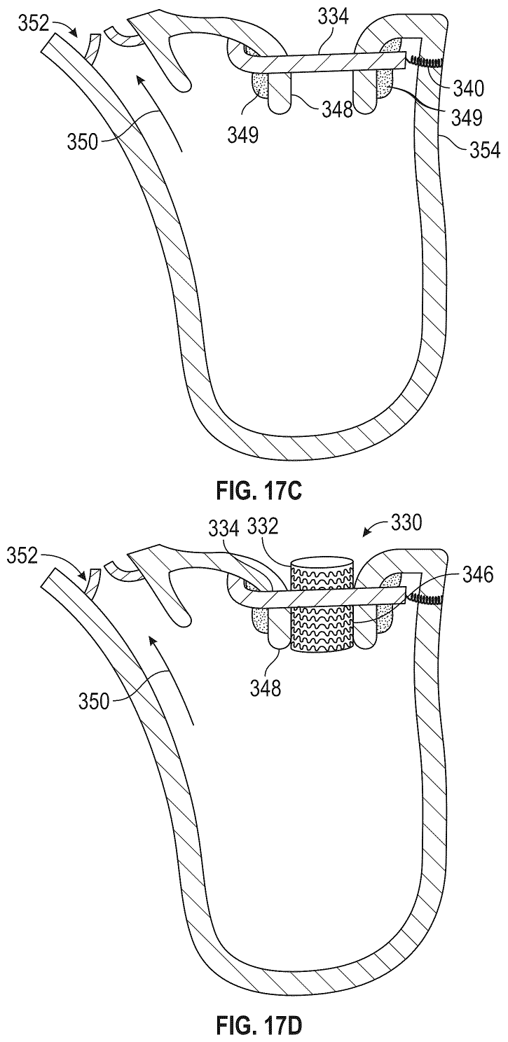

In examples, a configuration of the native valve 80 that includes calcification may impede the ability of a prosthetic valve to properly deploy to an implantation site. For example, calcification may impede the ability of a prosthetic valve to seal with a native valve. Calcification may impede the ability of a prosthetic valve to anchor to a native valve. Calcification may result in an obstruction of the left ventricular outflow tract (LVOT) 350 as marked in FIG. 17C, for example, based on implantation of a prosthetic valve to a native mitral valve having calcification. Calcification may produce other undesired effects for the implantation of a prosthetic valve or other treatment of a native valve or a heart.

For example, referring to FIG. 5, calcification 84 may be present underneath or radially outward of an outward facing surface of a leaflet 83 of a native heart valve 88. The outward facing surface may comprise a downstream side or surface. Such calcification 84 may impede the ability of anchors 17 as shown in FIG. 4 for example, from hooking around the native valve leaflets 83 to anchor to the native valve leaflets 83. The calcification 84 may block the anchors 17 from being positioned radially outward of the outward surface of the leaflet 83 in a desired manner.

Calcification 86 may be positioned radially inward of a leaflet 83. For example, calcification 86 may be positioned on an upstream side or surface or inward facing surface of a leaflet 83 or may protrude inward towards the flow channel between the leaflets 83. In examples, calcification 86 may be positioned on the annulus of the native heart valve 88 or may be positioned within the atrium of the heart. Such calcification 86 may impede the ability of a prosthetic valve from sealing or anchoring to the native valve. An irregular shaped annulus may result, which may impede the ability of a prosthetic valve to deploy to the native heart valve 88 in a desired manner. The calcification may be at the mitral valve, and may comprise mitral annular calcification (MAC).

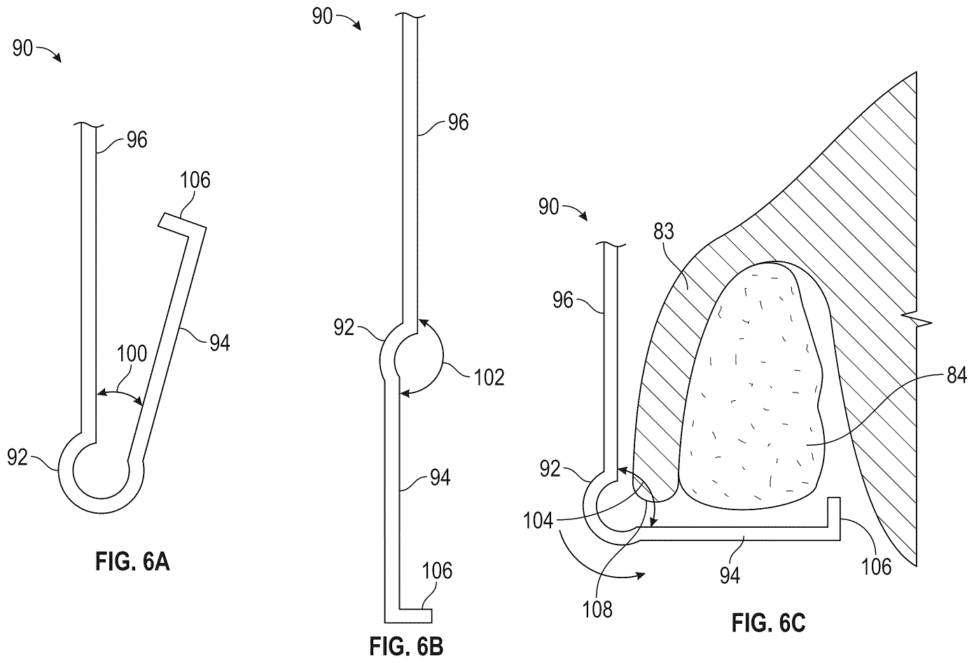

FIGS. 6A-6C illustrate an example of an anchor 90 that may be utilized in examples herein. The anchor 90 may include a hinge 92 forming a loop and a straight portion 94 configured to extend radially outward from the loop. The anchor 90 may include a coupling portion 96 that may be configured to couple to a support structure configured to support one or more prosthetic valve leaflets, for example a support structure 15 shown in FIG. 1A or another form of support structure. At least one anchor 90 may be coupled to the support structure 15. The anchor 90 may be utilized with a prosthetic valve having one or more prosthetic valve leaflets configured to be positioned in a flow channel, as shown in FIG. 2 for example. The anchor 90 may be configured as an arm or may have another configuration in examples.

The coupling portion 96 may be configured to couple to a frame of the support structure, for example at a distal portion 98 of a support structure or frame as marked in FIG. 2. The coupling portion 96 may extend distally from the distal portion 98 of the support structure to the hinge 92 in examples. The coupling portion 96 may comprise an arm or may have another configuration in examples.

The hinge 92 may form a loop that may protrude radially inward as shown in FIG. 6A and may have a semi-circle shape. The loop shape of the hinge 92 may allow the straight portion 94 to rotate about the hinge 92 while the straight portion 94 maintains a straight shape. The hinge 92 may be positioned between the straight portion 94 and the coupling portion 96 that is configured to couple the hinge 92 to the support structure.

The hinge 92 may allow the straight portion 94 to extend at a variety of angles relative to the support structure and relative to a central axis 61 of a prosthetic valve as shown in FIG. 2 for example. As shown in FIG. 6A, the straight portion 94 may extend at an acute angle 100, which may be between 0 and 90 degrees, or may extend to a straight angle 102 or greater as shown in FIG. 6B. The angle 104 may be a perpendicular angle as shown in FIG. 6C for example.

The straight portion 94 may comprise a linear portion that may extend from the hinge 92 to a tip 106 of the anchor 90. The straight portion 94 may be positioned between the hinge comprising the loop and the tip 106. The tip 106 may be angled relative to the straight portion 94.

Prior to deployment, the anchor 90 may be held in an elongated or straight angle position as shown in FIG. 6B. The anchor 90 may extend with the tip 106 distal of the hinge 92. Upon deployment, the anchor 90 may be released such that the straight portion 94 rotates about the hinge 92 in a proximal direction.

For example, referring to FIG. 6C, the straight portion 94 has rotated proximally to reduce the angle 104 between the coupling portion 96 and the straight portion 94. The straight portion 94 may be configured to extend radially outward of a native valve leaflet 83 and may extend over a distal tip 108 of the leaflet 83. The straight portion 94 may contact calcification 84, which may be positioned radially outward of the leaflet 83. The straight portion 94 may extend along the calcification 84 to anchor to the calcification 84 and resist a proximal force applied to the anchor 90 and the support structure. In examples, the tip 106 may engage the calcification 84 to anchor to the calcification 84.

The hinge 92 may be biased to rotate the straight portion 94 proximally. For example, the straight portion 94 is shown extending perpendicular in FIG. 6C, yet the straight portion 94 may continue to rotate proximally to a configuration as shown in FIG. 6A depending on the shape of the calcification 84 and the native valve. Varied angles between the straight portion 94 and the coupling portion 96 may result.

Features of the examples of FIGS. 1A-6C may be utilized solely or in combination with any other example disclosed herein.

In examples, other forms of anchors may be utilized.

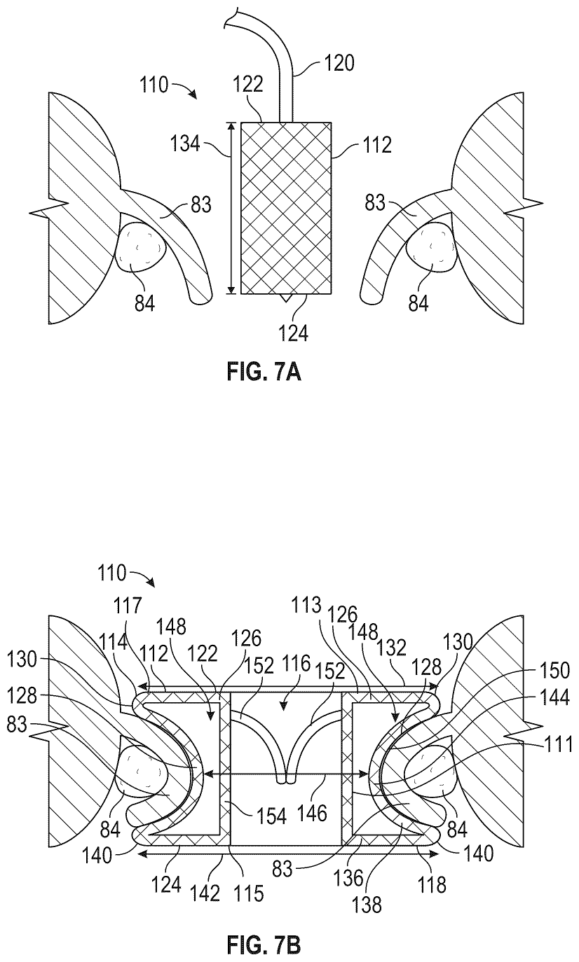

FIGS. 7A-7B illustrate an example of a prosthetic valve 110 configured to include a support structure 112 including a proximal anchor (marked in FIG. 7B) comprising a proximal or first flange 114 configured to extend radially outward from a passageway or flow channel 116 and a distal anchor (marked in FIG. 7B) comprising a distal or second flange 118.

Referring to FIGS. 7A and 7B, the support structure 112 may include an inner support stent 111 having an inlet end portion 113 and an outlet end portion 115. The support structure 112 may include an outer conformable structure 117 for conforming to calcification on the calcified native mitral valve for enhancing sealing and anchoring to the calcified native mitral valve. The outer conformable structure 117 may surround the inner support stent 111. The inner support stent 111 and/or the outer conformable structure 117 may comprise a mesh that may be in a form of a weave or may have another configuration. The mesh may allow the support structure 112 to move and conform to a shape of a native valve, including a native valve having calcification. For example, referring to FIG. 7A, the support structure 112 may be in an undeployed or unexpanded configuration and may have a cylindrical configuration. The flanges 114, 118 may not protrude from the support structure 112 in such a configuration or may not protrude as fully as shown in FIG. 7B. The flanges 114, 118 may be made of the mesh in examples. The support structure 112 may be held in a cylindrical configuration in the undeployed or unexpanded configuration to reduce profile or outer diameter of the support structure 112 upon passage through vasculature of an individual. The flanges 114, 118 may be held in a retracted configuration in the cylindrical configuration. The support structure 112 may be covered with a component of a delivery apparatus (e.g., a capsule or other component) or may be exposed upon passage through the vasculature.

The support structure 112 may be configured to expand radially outward to an expanded configuration. The outer conformable structure 117 may form the flanges 114, 118. The flanges 114, 118 may be configured to move radially outward in the expanded configuration. The support structure 112 may be biased to expand radially outward in examples. For example, the support structure 112 may be made of a shape memory material that may expand radially outward upon full or partial release from a delivery apparatus 120. The shape memory material may comprise Nitinol (NiTi) or may comprise another form of shape memory material in examples. In examples, the support structure 112 may be configured to be compressed axially to cause the support structure 112 to expand radially outward. A delivery apparatus 120, for example, may be configured to compress the proximal end 122 and the distal end 124 of the support structure 112 together to cause the support structure 112 to expand radially outward.

FIG. 7B illustrates a cross sectional view of the support structure 112 having expanded radially outward to a deployed or expanded configuration. The first flange 114 may extend radially outward and may form a disk. The disk may extend circumferentially about the flow channel 116. The disk may be positioned on a proximal side or an atrial side of the native heart valve and may resist a distal or ventricular force applied to the support structure 112.

The disk of the first flange 114 may include a first end portion 126 and a second end portion 128 and a protruding portion 130 configured to protrude radially outward from the first end portion 126 and the second end portion 128. The protrusion of the protruding portion 130 may define the diameter 132 of the disk. The first end portion 126 and the second end portion 128 may be configured to move towards each other when the support structure 112 expands radially outward from the configuration shown in FIG. 7A to the expanded configuration shown in FIG. 7B. The movement between the end portions 126, 128 may be axial with the protruding portion 130 extending radially outward from the end portions 126, 128. As such, an axial length 134 (marked in FIG. 7A) of the support structure 112 may decrease while the diameter 132 of the support structure 112 may increase. The protruding portion 130 may comprise a loop of material that forms an outer wall 150 of the outer conformable structure 117.

The second flange 118 may be configured similarly as the first flange 114. The second flange 118, for example, may extend radially outward and may form a disk. The disk may extend circumferentially about the flow channel 116. The disk may be positioned on a distal side or a ventricular side of the native heart valve and may resist a proximal or atrial force applied to the support structure 112. The disk may similarly include a first end portion 136 and a second end portion 138 and a protruding portion 140 configured to protrude radially outward from the first end portion 136 and the second end portion 138. The protrusion of the protruding portion 140 may define the diameter 142 of the disk. The first end portion 136 and the second end portion 138 may be configured to move towards each other when the support structure 112 expands radially outward from the configuration shown in FIG. 7A to the expanded configuration shown in FIG. 7B. The movement between the end portions 136, 138 may be axial with the protruding portion 140 extending radially outward from the end portions 136, 138.

A central portion 144 may be positioned between the first flange 114 and the second flange 118 and may comprise a reduced diameter portion of the support structure 112. The central portion 144 for example may have a lower diameter 146 than the diameter 132 of the first flange 114 and the diameter 142 of the second flange 118. The central portion 144 may comprise a waist of the support structure 112 that may be bowed inward from the protruding portions 130, 140 of the respective first flange 114 and second flange 118.

In examples, the central portion 144 may be configured to conform to a shape of a native valve. The central portion 144 may be configured to accommodate calcification or other shapes of a native valve. In examples, the central portion 144 may comprise a curved portion that may curve radially inward from the protruding portions 130, 140 of the respective first flange 114 and second flange 118. The central portion 144 may have other shapes in examples.

One or more of the central portion 144 or flanges 114, 118 may be configured to conform to a shape of the native valve to provide a seal with the native valve. In examples, all or a portion of the support structure 112 may include a sealing skirt or may be otherwise configured to provide a seal with the native valve.

In examples, the support structure 112 may include a cavity 148 that may extend between an outer wall 150 of the outer conformable structure 117 and the flow channel 116 of the support structure 112. In examples, the interior of the support structure 112 may be filled and may lack a cavity 148.

The support structure 112 may be coupled to one or more prosthetic valve leaflets 152 that may be positioned within the flow channel 116. The support structure 112 and particularly the inner support stent 111 may be configured to support the one or more prosthetic valve leaflets 152. The prosthetic valve leaflets 152 may be coupled to an inner wall 154 of the inner support stent 111 and may extend radially inward from the inner wall 154.

In examples, the support structure 112 and inner support stent 111 may comprise a dock that may be configured to engage with an insert such as a leaflet support body that may retain the prosthetic valve leaflets 152. In such an example, the support structure 112 may be deployed initially, with the insert deployed in a subsequent action that may engage with the inner support stent 111.

The support structure 112 may beneficially be configured to conform to a shape of the native valve including calcification 84. The calcification 84 may be positioned radially outward of the native valve leaflets 83 or may be positioned radially inward or atrial of the native valve leaflets 83. The first flange 114 or second flange 118 may be configured to anchor to and conform to calcification of a native valve or another portion of a native valve.

The prosthetic valve 110 may be utilized with a mitral or a tricuspid valve of a heart, or another location for deployment as desired.

Features of the examples of FIGS. 7A-7B may be utilized solely or in combination with any other example disclosed herein.

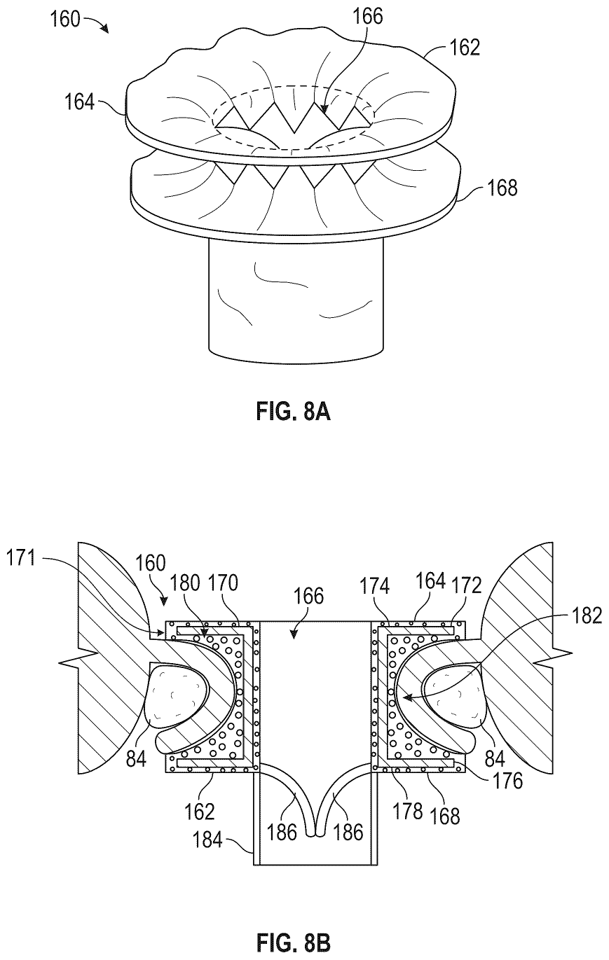

FIGS. 8A-8B illustrate an example of a prosthetic valve 160 including a support structure 162 including a proximal anchor comprising a proximal or first flange 164 configured to extend radially outward from a flow channel 166 and a distal anchor comprising a distal or second flange 168 configured to extend radially outward from the flow channel 166. The support structure 162 may be configured to support one or more prosthetic valve leaflets 186 that may be positioned within the flow channel 166.

The support structure 162 may include an inner support stent including a frame 170 (marked in FIG. 8B) that may be configured to form the first flange 164 and the second flange 168 and may extend between the first flange 164 and the second flange 168.

The portion of the frame 170 comprising the first flange 164 may be configured to be positioned in an undeployed or compressed configuration with a first end portion 172 positioned proximal of a second end portion 174. Upon moving to a deployed configuration or an expanded configuration the first end portion 172 may move radially outward from the second end portion 174 to a position as shown in FIGS. 8A and 8B. The first flange 164 may extend horizontally relative to a central portion of the frame 170. Similarly, the portion of the frame 170 comprising the second flange 168 may be configured to be positioned in an undeployed or compressed configuration with a first end portion 176 positioned distal of a second end portion 178. Upon moving to a deployed configuration or an expanded configuration the first end portion 176 may move radially outward from the second end portion 178 to a position as shown in FIGS. 8A and 8B. The second flange 168 may extend horizontally relative to a central portion of the frame 170. The first flange 164 and the second flange 168 accordingly may be retracted with the support structure 162 in a cylindrical configuration and configured to move radially outward in an expanded configuration.

The first flange 164 may form a disk, and the second flange 168 may form a disk. Each disk may extend circumferentially about a flow channel 166 as shown in FIG. 8A for example. The disk of the first flange 164 may be positioned on a proximal side or an atrial side of the native heart valve and may resist a distal or ventricular force applied to the support structure 162. The disk of the second flange 168 may be positioned on a distal side or a ventricular side of the native heart valve and may resist a proximal or atrial force applied to the support structure 162.

An outer conformable structure may be provided to conform to calcification on the calcified native mitral valve for enhancing sealing and anchoring to the calcified native mitral valve. For example, the frame 170 may be covered in a material comprising the outer conformable structure that may be configured to form a seal with the native valve and may form an outer surface 171 of the prosthetic valve 160. The material may comprise a sealing skirt in examples or another form of material. A conformable or compliant body such as a padding 180 may be positioned between the material and the frame 170 that may be configured to conform to a shape of the native valve. For example, a padding 180 may comprise a compressible cloth or compressible foam that may be configured to conform to a shape of the native valve. The padding 180 may comprise a shape memory material (e.g. a shape memory foam) in examples. The padding 180 may be positioned on one or more of the flanges 164, 168, or a central portion 182 of the support structure 162 positioned between the flanges 164, 168. As such, the support structure 162 and flanges 164, 168 may be configured to anchor to and conform to a shape of a native valve, which may include calcification.

In examples, the second flange 168 may comprise a plurality of distal anchors, such as paddles or another form of anchor, that may be positioned distal or ventricular of the native valve.

In examples, the support structure 162, and particularly the inner support stent may include a distal portion 184 that may protrude distally from the second flange 168. The prosthetic valve leaflets 186 may be positioned within the distal portion 184 or may be positioned otherwise with respect to the support structure 162. In examples, the distal portion 184 may comprise a cylindrical portion distally protruding from the second flange 168 or may have another configuration as desired.

The prosthetic valve 160 may be utilized with a mitral or a tricuspid valve of a heart, or another location for deployment as desired.

Features of the examples of FIGS. 8A-8B may be utilized solely or in combination with any other example disclosed herein.

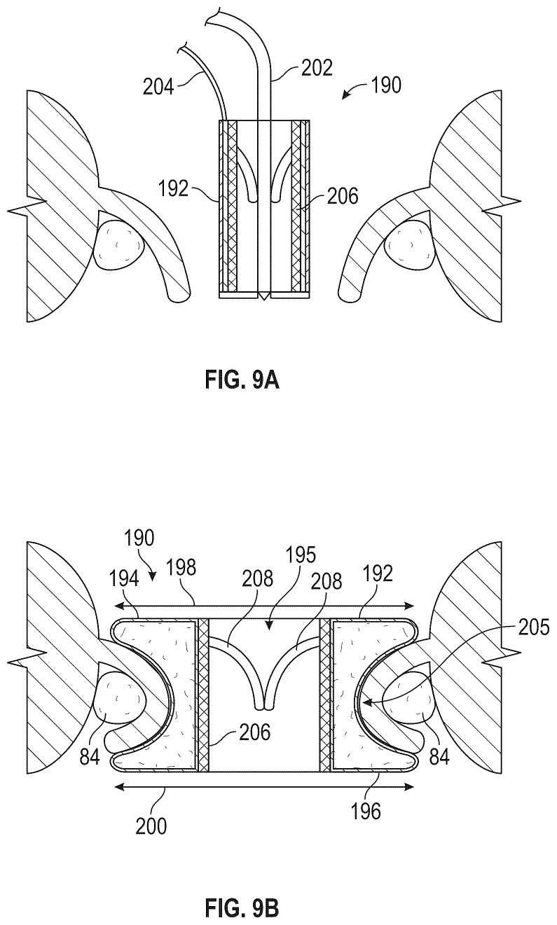

FIGS. 9A-9B illustrate an example of a prosthetic valve 190 including a support structure 192 including a proximal anchor comprising a proximal or first flange 194 (marked in FIG. 9B) configured to extend radially outward from a flow channel 195 and a distal anchor comprising a distal or second flange 196 (marked in FIG. 9B) configured to extend radially outward from the flow channel 195. The support structure 192 may be configured to support one or more prosthetic valve leaflets 208 that may be configured to be positioned in the flow channel 195.

The first flange 194 may comprise an inflatable body, and the second flange 196 may comprise an inflatable body in examples. The support structure 192 may comprise a single inflatable body that may join the first flange 194 to the second flange 196 in examples (as shown in FIG. 9B) or may comprise a plurality of inflatable bodies. The inflatable body may be positioned upon an inner support stent or frame 206 of the support structure 192 in examples. The inflatable body may comprise an outer conformable structure or bladder configured to be filled with fluid to be inflated to increase the diameter 198 of the first flange 194 and the diameter 200 of the second flange 196 in examples. The inflatable body may comprise a chamber. The bladder may comprise a silicone bladder or may have another configuration in examples.

For example, referring to FIG. 9A, the support structure 192 may be in a compressed or undeployed configuration and may be advanced with a delivery apparatus 202 to the implantation site. The inflatable body may be in an uninflated configuration. A fluid conduit 204 may extend to the inflatable body to fill the inflatable body in examples.

Upon the support structure 192 being positioned in a desired location, the inflatable body may be filled via the fluid conduit 204. Referring to FIG. 9B, the first flange 194 may expand radially outward to form a disk positioned on a proximal or atrial side of the native valve, and the second flange 196 may expand radially outward to form a disk positioned on a distal or ventricular side of the native valve. The flanges 194, 196 accordingly may each be retracted in a cylindrical configuration as shown in FIG. 9A, and may be configured to move radially outward in an expanded configuration. The flanges 194, 196 may be filled with fluid to conform to a shape of the native valve, which may include calcification 84. The flanges 194, 196 may anchor to the calcification 84. A central portion 205 of the support structure 192 between the first flange 194 and the second flange 196 may similarly be filled with fluid to conform to a shape of the native valve. The support structure 192 may seal with the native valve to reduce fluid flow around the support structure 192 (e.g., paravalvular leakage).

In examples, a fill material that may be utilized may comprise a fluid such as saline or other forms of fluid. The fill material may be a liquid, foam, epoxy, gas, or other material. The fill material may be liquid to result in a hydraulic inflation of the inflatable body. A fill material in the form of a gas may comprise carbon dioxide or helium, among other forms of gas.

In embodiments, the fill material may comprise a hardenable material. The fill material may be configured to harden over time to enhance the sealing of the outer conformable structure. The hardenable material that may be introduced into an inflatable body at a first, relatively low viscosity and converted to a second, relatively high viscosity. Viscosity enhancement may be accomplished through a variety of UV initiated or catalyst initiated polymerization reactions, or other chemical system. The end point of the viscosity enhancing process may result in a hardness anywhere from a gel to a rigid structure, depending on the desired performance.

A hardenable material may comprise an epoxy. The epoxy may be hardened by mixing materials that harden when combined. The hardening catalyst may be delivered during implantation or later. The hardenable material may be biocompatible and able to conform to the shape of the local native valve. In embodiments, the hardenable material may be bioresorbable.

In embodiments, the fill material may be radiopaque for visualization during implantation. A radiopaque material may be added during filling, as part of a hardening process for example.

In embodiments, the fill material may comprise a gel or a foam, which may be biocompatible, and may be configured to harden over time. A gel or foam may be inserted into the sealing body, or may be provided in capsules that dissolve upon implantation to allow for expansion.

In embodiments, a gel may be utilized that may be made via polymer precipitation from biocompatible solvents. Various siloxanes may be utilized as inflation gels as well. Other gel systems that may be utilized may include phase change systems that gel upon heating or cooling from their initial liquid or thixotropic state. Gels may also comprise thixotropic material that undergo sufficient shear-thinning so that they may be readily injected through a fluid conduit yet are also gel-like at zero or low shear rates.

In embodiments, a fill material may contain a foaming agent. The foaming agent may generate pressure within the inflatable body.

Any of the fill materials disclosed herein may be biocompatible in embodiments, and may be bioresorbable if desired. A bioresorbable sealing body may improve sealing through tissue adhesion with the native valve.

In examples, the inflatable body of the support structure 192 may be positioned upon an inner support stent or frame 206 that may support the prosthetic valve leaflets 208 within the flow channel 195.

The prosthetic valve 190 may be utilized with a mitral or a tricuspid valve of a heart, or another location for deployment as desired.

Features of the examples of FIGS. 9A-9B may be utilized solely or in combination with any other example disclosed herein.

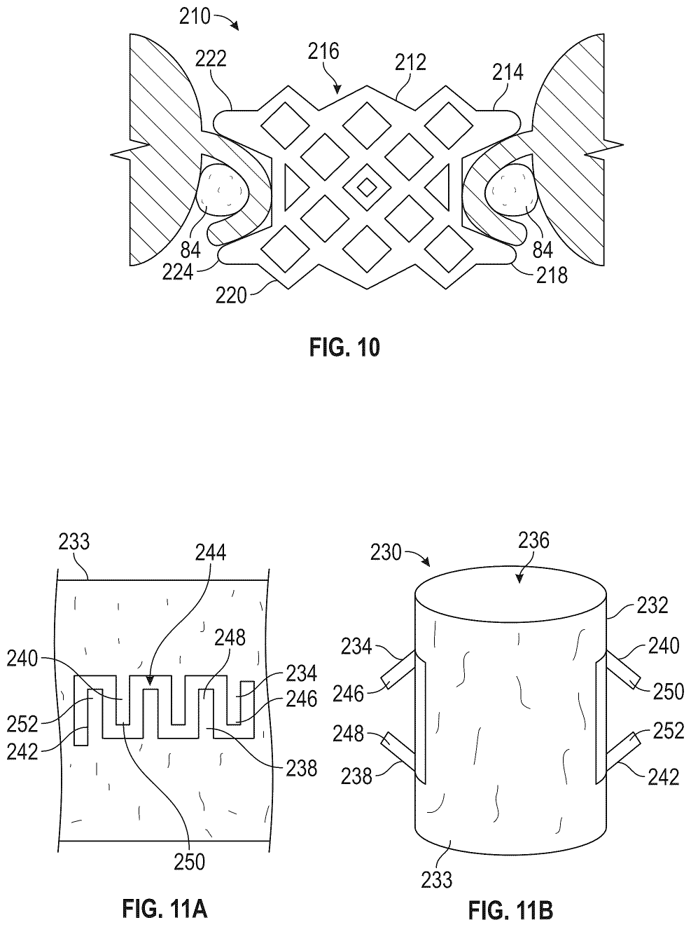

FIG. 10 illustrates an example of a prosthetic valve 210 including a support structure 212 including a proximal anchor comprising a proximal or first flange 214 configured to extend radially outward from a flow channel 216 and a distal anchor comprising a distal or second flange 218 configured to extend radially outward from the flow channel 216.

The support structure 212 may include an inner support stent or frame 220 that may support one or more prosthetic valve leaflets (not shown) in the flow channel 216. The frame 220 is visible in FIG. 10 and other components of the prosthetic valve 210 are excluded from view for clarity in FIG. 10. The frame 220 may include a proximal ridge 222 that may comprise the proximal or first flange 214 and may include a distal ridge 224 that may comprise the distal or second flange 218. The first flange 214 and the second flange 218 may each be configured to anchor to the native valve, which may include calcification 84.

The first flange 214, for example, may be positioned on a proximal side or an atrial side of the native heart valve and may resist a distal or ventricular force applied to the support structure 212. The second flange 218 may be positioned on a distal side or a ventricular side of the native heart valve and may resist a proximal or atrial force applied to the support structure 212. In examples, a sealing body or outer conformable structure may be applied to the frame 220 to conform to calcification on the calcified native mitral valve for enhancing sealing and anchoring to the calcified native mitral valve.

The prosthetic valve 210 may be utilized with a mitral or a tricuspid valve of a heart, or another location for deployment as desired.

Features of the examples of FIG. 10 may be utilized solely or in combination with any other example disclosed herein.

FIG. 11B illustrates an example of a prosthetic valve 230 including a support structure 232 including a proximal anchor comprising a proximal or first flange 234 configured to extend radially outward from a flow channel 236 and a distal anchor comprising a distal or second flange 238 configured to extend radially outward from the flow channel 236. The support structure 232 may include a plurality of the flanges including an additional proximal flange 240 and a distal flange 242, and may include additional flanges.

The support structure 232 may be configured to support one or more prosthetic valve leaflets (not shown) that may be positioned within the flow channel 236.

FIG. 11A illustrates a flattened pattern of a frame 233 or inner support stent of the support structure 232 shown in FIG. 11B. The flanges 234, 238, 240, 242 may comprise cut out portions of a flat sheet of the frame. A channel 244 may be formed between the flanges 234, 238, 240, 242 due to the cut out of material from the frame 233. Each of the flanges 234, 238, 240, 242 may have respective ends 246, 248, 250, 252 that may be configured to protrude radially outward from the frame 233. The ends 250, 252 of the flanges 240, 242 may be angled towards each other (as shown in FIG. 11B) and the ends 248, 246 of the flanges 238, 234 may be angled towards each other (as shown in FIG. 11B). The flanges 234, 238, 240, 242 may each comprise barbs in examples.

The angle of the flanges 234, 238, 240, 242 may allow the proximal flanges 234, 240 to engage proximal or atrial portions of the native valve, and may allow the distal flanges 238, 242 to engage distal or ventricular portions of the native valve. The flanges 234, 238, 240, 242 may be configured to engage leaflets of the native valve, and may be configured to engage calcification of the native valve. In examples, a sealing body or outer conformable structure may be applied to the frame 233 to conform to calcification on the calcified native mitral valve for enhancing sealing and anchoring to the calcified native mitral valve.

The prosthetic valve 230 may be utilized with a mitral or a tricuspid valve of a heart, or another location for deployment as desired.

Features of the examples of FIGS. 11A-11B may be utilized solely or in combination with any other example disclosed herein.

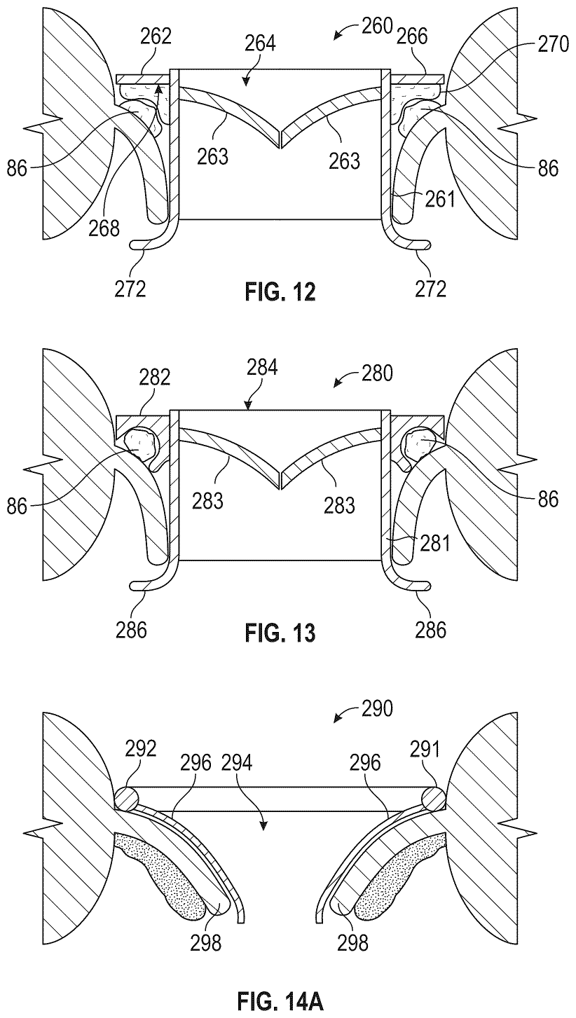

FIG. 12 illustrates an example of a prosthetic valve 260 having a support structure 261 including an atrial anchor 262 forming a ring about the flow channel 264. The support structure 261 may include a support stent. The support structure 261 may be for deployment on an upstream side of a native valve. The ring may comprise a disk that may extend circumferentially about the flow channel 264 and may extend radially outward from the flow channel 264. The support structure 261 may be configured to support one or more prosthetic valve leaflets 263 that may be positioned in the flow channel 264.

The ring may include an upper surface 266 configured to face in an atrial direction and a lower surface 268 configured to face opposite the upper surface 266 and in a ventricular direction. A compliant body 270 may be positioned on the lower surface 268 and may be configured to conform to a shape of the native valve. The compliant body 270, for example, may comprise an inflatable body or foam, other material that may be configured to conform to the shape of a native valve. An inflatable body may be filled with a hardenable material as disclosed herein. The compliant body 270 may be configured to conform to a shape of calcification 86 of the native valve. The atrial anchor 262 accordingly may anchor to calcification 86.

In examples, the compliant body 270 may comprise a sealing body configured to form a seal with the native valve. The seal may prevent fluid flow outside of the flow channel 264 (e.g., paravalvular leakage).

The ring may be configured to resist a distal or ventricular force applied to the prosthetic valve 260. The contact between the ring and the native valve or calcification 86 of the native valve for example may impede distal movement of the prosthetic valve 260.

The prosthetic valve 260 may deploy with the atrial anchor 262 angled upward or downward (in an undeployed configuration). The atrial anchor 262 may then extend radially outward to extend horizontally in a deployed configuration.

In examples, the prosthetic valve 260 may include one or more distal anchors 272 that may be configured to anchor the prosthetic valve 260 to the native valve from the ventricular side of the native valve. The distal anchors 272 may be configured similarly as any example of distal anchors disclosed herein.

The prosthetic valve 260 may be utilized with a mitral or a tricuspid valve of a heart, or another location for deployment as desired.

Features of the examples of FIG. 12 may be utilized solely or in combination with any other example disclosed herein.

FIG. 13 illustrates an example of a prosthetic valve 280 having a support structure 281 including an atrial anchor 282 forming a ring about the flow channel 284. The support structure 281 may include a support stent. The ring may extend radially outward from the flow channel 284. The support structure 281 may be configured to support one or more prosthetic valve leaflets 283 that may be positioned in the flow channel 284.

The ring may comprise a gasket that may be configured to conform to a shape of the native valve on the atrial side of the valve. For example, the gasket may be conformable and configured to conform to a shape of calcification 86 on the atrial side of the valve. The gasket accordingly may anchor to calcification 86. The gasket may comprise any form of compliant or conformable body disclosed herein. A foam or rubber material may be utilized in examples, among other forms of material.

The gasket may be configured to resist a distal or ventricular force applied to the prosthetic valve 280. The contact between the gasket and the native valve or calcification 86 of the native valve for example may impede distal or ventricular movement of the prosthetic valve 280.

In examples, the gasket may comprise a sealing body configured to form a seal with the native valve. The seal may prevent fluid flow outside of the flow channel 284 (e.g., paravalvular leakage).

The prosthetic valve 280 may further include one or more distal anchors 286 that may be configured to anchor the prosthetic valve 280 to the native valve from the ventricular side of the native valve. The distal anchors 286 may be configured similarly as any example of distal anchors disclosed herein.

The prosthetic valve 280 may be utilized with a mitral or a tricuspid valve of a heart, or another location for deployment as desired.

Features of the examples of FIG. 13 may be utilized solely or in combination with any other example disclosed herein.

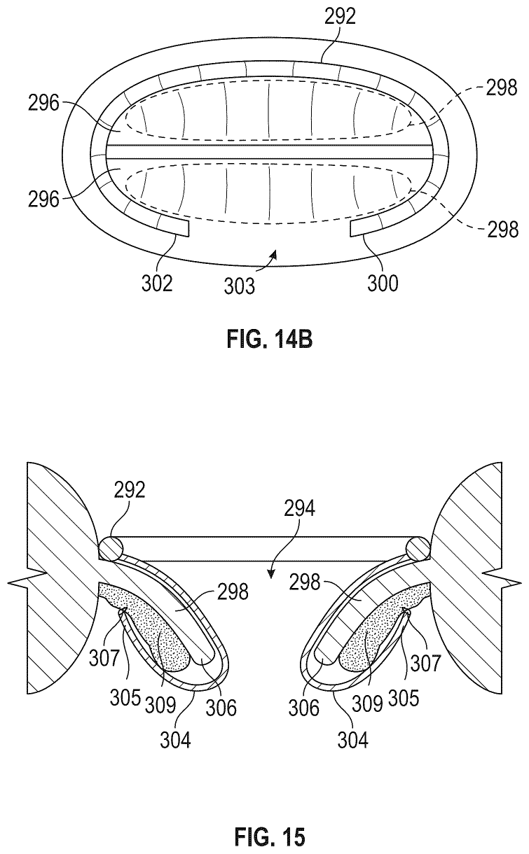

FIGS. 14A-14B illustrate an example of a prosthetic valve 290 including a support structure 291 having an atrial anchor 292 forming a ring about the passageway or flow channel 294. The ring may have a tubular shape. The support structure 291 may be configured to support one or more prosthetic valve leaflets 296 that may be positioned in the flow channel 294.

One or more prosthetic valve leaflets 296 may be coupled to the ring. The one or more prosthetic valve leaflets 296 may be configured to overlay one or more native valve leaflets 298 of the native valve. The one or more prosthetic valve leaflets 296 may overlay an upstream side of the one or more native valve leaflets 298. The one or more prosthetic valve leaflets 296 may be configured to provide coaptation with each other (e.g., during systole) and may be configured to open (e.g., during diastole).

Referring to FIG. 14B, the atrial anchor 292 may extend about the atrial side of the native valve and may have a crescent shape with end portions 300, 302 of the atrial anchor 292 separated from each other with a gap 303. The end portions 300, 302 may be repositionable to accommodate a size of the native valve. The atrial anchor 292 may be anchored in position due to a radially outward expansion force of the atrial anchor 292 or other forms of anchors may be utilized to secure the atrial anchor 292 in position. Other forms of anchors may include barbs, screws, adhesives, or other methods of anchoring. The prosthetic valve 290 may be utilized with a mitral or a tricuspid valve of a heart, or another location for deployment as desired.

Features of the examples of FIGS. 14A-14B may be utilized solely or in combination with any other example disclosed herein.

The configuration of the prosthetic valve leaflets may be varied in examples. For example, FIG. 15 illustrates a configuration of the prosthetic valve leaflets 304 extending around the native valve leaflets 298 to be positioned on the ventricular or distal or downstream facing surfaces of the native valve leaflets 298. The prosthetic valve leaflets 304 may hook around or wrap around the tips 306 of the native valve leaflets 298. The prosthetic valve leaflets 304 may overlay the ventricular or distal or downstream side of the native valve leaflets 298 (and the calcification in examples). The prosthetic valve leaflets 304 may wrap around the native valve leaflets 298 and cover an upstream side and a downstream side of the leaflets 298. The prosthetic valve leaflets 304 may include distal end portions 305 that have a barb 307 or other form of anchor for anchoring to the calcification 309 on the ventricular or distal or downstream side of the native valve leaflets 298. The barb 307 may engage the calcification 309 to secure the position of the prosthetic valve leaflets 304 upon the native valve leaflets 298.

Features of the example of FIG. 15 may be utilized solely or in combination with any other example disclosed herein.

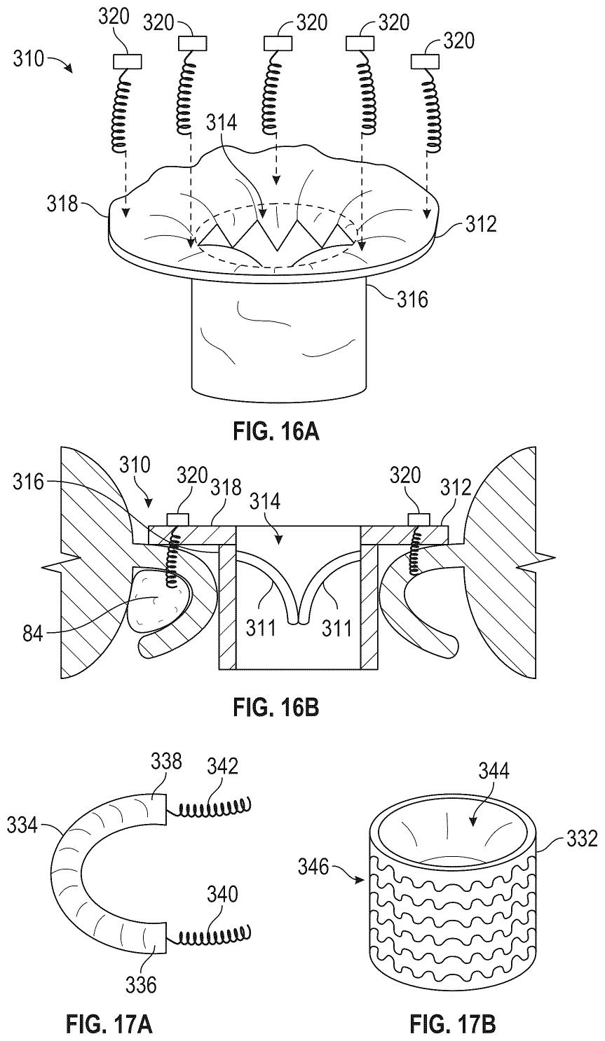

FIGS. 16A-16B illustrate an example of a prosthetic valve 310 including a support structure 318 having an atrial anchor 312 forming a ring about the flow channel 314. The atrial anchor 312 may comprise a flange configured as a disk extending radially outward from the flow channel 314 at a proximal end portion 316 of the a support structure 318, and extending circumferentially about the flow channel 314. The a support structure 318 may be configured to support one or more prosthetic valve leaflets 311 that may be positioned in the flow channel 314.

In examples, one or more penetrating bodies 320 may be configured to pass through the ring to anchor the a support structure 318 to the native valve. The penetrating bodies 320 may comprise screws or may have another form in examples. In examples, the penetrating bodies 320 may have other forms such as barbs or expandable clips or other forms as desired. The penetrating bodies 320 may be inserted upon the proximal or atrial side of the ring and may be positioned circumferentially about the flow channel 314.

In examples, the one or more penetrating bodies 320 may be configured to anchor to calcification 84 of a native valve, as shown in FIG. 16B for example.

The prosthetic valve 310 may be utilized with a mitral or a tricuspid valve of a heart, or another location for deployment as desired.

Features of the examples of FIGS. 16A-16B may be utilized solely or in combination with any other example disclosed herein.

FIGS. 17A-17D illustrates an example of a prosthetic valve system 330 (marked in FIG. 17D) configured to be deployed to a native valve. The prosthetic valve system 330 may include a valve body or support structure 332 (marked in FIG. 17B) and a dock 334 (marked in FIG. 17A). The dock 334 may be configured to dock with the support structure 332.

Referring to FIG. 17A, the dock 334 may comprise an elongate body having a first end portion 336 and a second end portion 338 and configured to form a crescent shape. The first end portion 336 may include a first penetrating body 340 configured to anchor to heart tissue. The second end portion 338 may include a second penetrating body 342 configured to anchor to heart tissue.

The first penetrating body 340 and second penetrating body 342 may each comprise a screw or may have another configuration as desired (e.g., a barb or expandable clip, or another configuration). The first penetrating body 340 and second penetrating body 342 may each be configured to anchor to tissue, with the dock 334 forming a loop for receiving the support structure 332.

Referring to FIG. 17B, the support structure 332 may support one or more prosthetic valve leaflets (not shown) and may surround a flow channel 344. The one or more prosthetic valve leaflets may be configured to be positioned in the flow channel 344. The support structure 332 may support the prosthetic valve leaflets. The support structure 332 may include an outer surface 346 that may comprise a sealing surface configured to form a seal with the native valve, including the native valve leaflets. The outer surface 346 may comprise a cloth surface in examples or may have another configuration.

FIG. 17C illustrates a deployed configuration of the dock 334. The dock 334 may extend over a native valve leaflet 348 (e.g., an anterior leaflet) that may be positioned proximate a left ventricular outflow tract (LVOT) 350. The dock 334 may extend around a radially outward surface of the leaflet 348. The dock 334 may pull the leaflet 348 away from the LVOT 350 to reduce the possibility of the leaflet 348 extending radially outward undesirably and blocking or otherwise obstructing the LVOT 350 and fluid flow to the aortic valve 352. As such, the native valve leaflet 348 may be anchored to the posterior wall 354 to reduce the possibility of the leaflet 348 obstructing the LVOT 350. The anchoring of the native valve leaflet 348 may be utilized to address the presence of calcification 349 that may be present proximate the native valve.

The first and second penetrating bodies 340, 342 may have sufficient anchoring strength to the tissue wall such that the anchors resist a radial force (e.g., a systolic force resisted by native chords).

Referring to FIG. 17D, with the dock 334 in position, the support structure 332 may be deployed to the dock 334. The support structure 332 may be deployed such that the leaflet 348 is positioned between the outer surface 346 of the support structure 332 and the dock 334.

The prosthetic valve system 330 may be utilized with a mitral or a tricuspid valve of a heart, or another location for deployment as desired.

Features of the examples of FIGS. 17A-17D may be utilized solely or in combination with any other example disclosed herein.

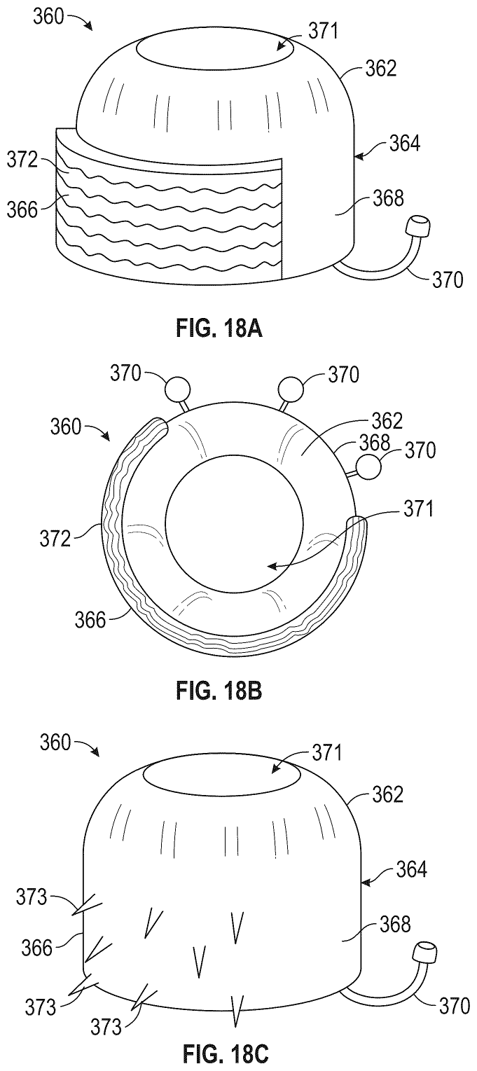

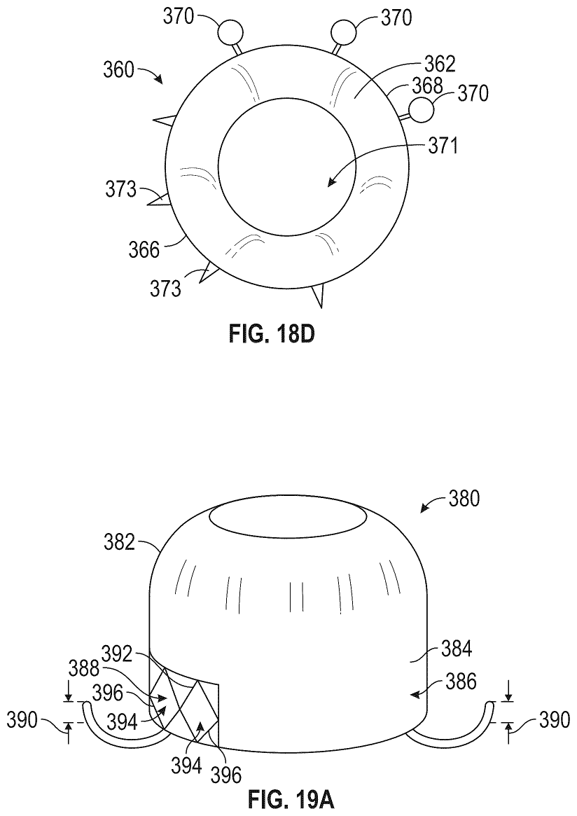

FIGS. 18A-18D illustrate an example of a prosthetic valve 360 including a valve body or support structure 362 including an outer perimeter 364 including a first portion 366 forming at least a quarter of the outer perimeter 364 and a second portion 368 forming a remainder of the outer perimeter 364. The support structure 362 may include one or more distal anchors 370 that protrude from the second portion 368 and may lack distal anchors that protrude from the first portion 366. The support structure 362 may be configured to support one or more prosthetic valve leaflets (not shown) that are configured to be positioned in the flow channel 371.

In examples, the first portion 366 may comprise a posterior portion of the support structure 362. The first portion 366 may comprise the posterior portion because calcification may be more likely to occur on a posterior side of a native valve (e.g., a mitral valve). Excluding or lacking the distal anchors on the posterior side may reduce the possibility of distal anchors 370 improperly anchoring to such calcification. The second portion 368, or portion including distal anchors 370, may include distal anchors 370 to couple with the native valve leaflets. The distal anchors 370 may be configured to hook over a distal tip of a leaflet of a native valve. The native valve leaflets may lack calcification or may have calcification in examples. In examples, the position of the first and second portions may be varied as desired. The distal anchors 370 may be configured similarly as any example of distal anchor disclosed herein.

Referring to FIGS. 18A and 18B, the first portion 366 may include a sealing surface 372 that may be configured to form a seal with a native valve, including calcification of the native valve. The sealing surface 372 may be compliant and configured to conform to the shape of the native valve, such as the shape of calcification. A compliant and conformable body may be utilized. The sealing surface 372 may comprise a sealing cloth or padded cloth that may be compliant or conformable and may conform to the shape of the native valve.

In examples, the first portion 366 may form a lesser or greater proportion of the outer perimeter 364. For example, the first portion 366 may comprise at least a half of the proportion of the outer perimeter 364 in examples. A greater proportion may be utilized in examples. For example, as shown in FIGS. 18A and 18B, the first portion may comprise at least two-thirds of the proportion of the outer perimeter 364. Varied proportions may be utilized in examples.

Referring to FIGS. 18C and 18D, in examples, the first portion 366 may include one or more barbs 373 or other form of protrusion protruding from the outer perimeter 364. The barbs 373 may be for anchoring with a native valve. The barbs 373 may be spaced from each other on the first portion 366 and may protrude radially outward from the support structure 362. The barbs 373 may be configured to pierce a surface of a native valve (e.g., an inward facing wall or an upstream side of the native valve leaflets) to provide anchoring. The barbs 373 may be configured to penetrate calcification in examples (i.e., for replacing a calcified valve). In various embodiments, the barbs may point toward the inflow end of the support structure, toward the outflow end of the support structure or directly outwardly. The barbs may also be angled circumferentially such that rotation of the support structure causes the barbs to penetrate tissue and/or calcification.

In examples, a combination of barbs 373 or other forms of protrusions and a sealing surface 372 (e.g., a compliant or conformable body) may be provided. The barbs 373, for example, may protrude from the sealing surface 372 upon a radially inward pressure being applied to the sealing surface 372. The barbs 373 may protrude from the outer surface of the sealing surface 372 for anchoring. The sealing surface 372 may compress inward for sealing, with the barbs 373 providing anchoring with the native valve. Other combinations may be utilized in examples.

The prosthetic valve 360 may be utilized with a mitral or a tricuspid valve of a heart, or another location for deployment as desired.

Features of the examples of FIGS. 18A-18B may be utilized solely or in combination with any other example disclosed herein.

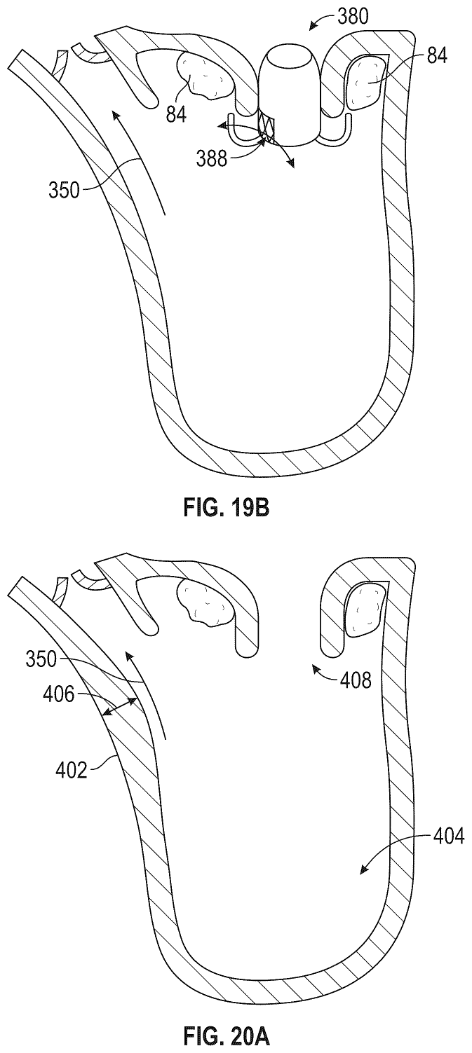

FIGS. 19A-19B illustrate an example of a prosthetic valve 380 including a valve body or support structure 382 including a sealing body 384 having a first portion 386 and a second portion 388, the first portion 386 impeding fluid flow at a height 390 of the support structure 382 and the second portion 388 being at the height 390 of the first portion 386 and being at a circumferentially offset position from the position of the first portion 386. The second portion 388 may be recessed to allow for fluid flow through at the circumferentially offset position. The support structure 382 may support one or more prosthetic valve leaflets (not shown) that may be configured to be positioned in the flow channel 389.

As such, referring to FIG. 19A, the recessed second portion 388 of the sealing body 384 may be recessed from a frame 392 of the support structure 382. The recessed second portion 388 may expose openings 394 of the frame 392 that may be positioned between struts 396 of the frame 392. The openings 394 may be bound by the plurality of struts 396. The sealing body 384 at the second portion 388 does not cover at least one of the plurality of openings 394 to allow for fluid flow through. Fluid accordingly may flow through at least one of the openings 394 at the second portion 388 due to the second portion 388 being recessed.

The sealing body 384 may comprise a sealing skirt or may have other forms in examples.

In examples, a portion of the frame 392 may be recessed at the second portion 388 to allow for fluid flow through.

In examples, the second portion 388 may comprise an anterior portion of the support structure 382. The second portion 388 may comprise an anterior portion to allow for fluid flow therethrough to allow for increase fluid flow at the left ventricular outflow tract (LVOT) 350. For example, referring to FIG. 19B, the second portion 388 may be positioned to allow for fluid flow therethrough, and to the LVOT 350. The second portion 388 may be at a height that is distal of the prosthetic valve leaflets of the prosthetic valve 380, to allow for fluid flow through the second portion 388 and for operation of the prosthetic valve leaflets.

The prosthetic valve 380 may be utilized with a mitral or a tricuspid valve of a heart, or another location for deployment as desired.

Features of the examples of FIGS. 19A-19B may be utilized solely or in combination with any other example disclosed herein.

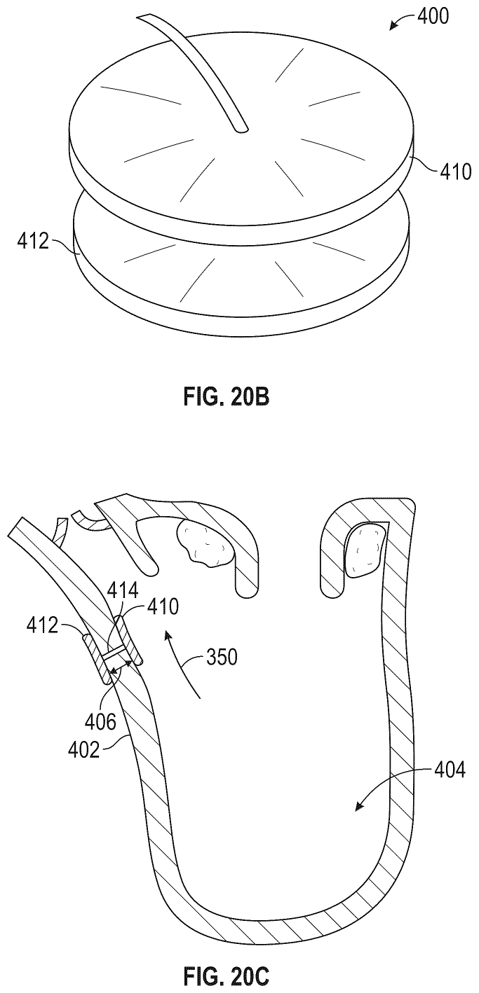

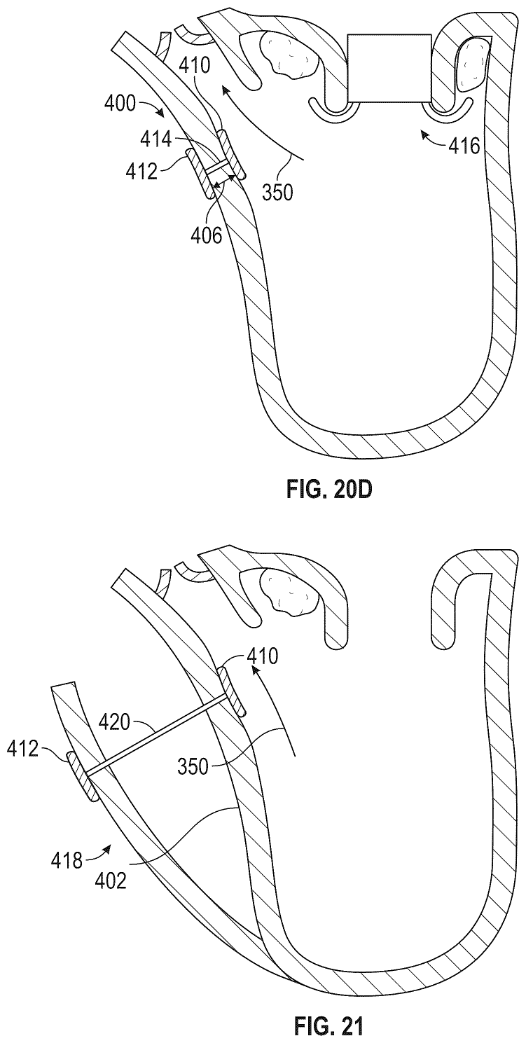

In examples, a compression system 400 (marked in FIG. 20B) may be utilized to compress a portion of a heart. Referring to FIG. 20A, in individuals the portion of the interventricular septum 402 between the left ventricle 404 and the right ventricle has a thickness 406. The thickness 406 may be desirably reduced to increase a size of the LVOT 350, to reduce the possibility of obstruction or other blockage of the LVOT 350. For example, a prosthetic heart valve implanted into a calcified mitral valve 408 may increase the possibility of the LVOT 350 being obstructed. The thickness 406 may be reduced to reduce a protrusion of the interventricular septum 402 into the LVOT 350. Such a reduction in thickness 406 may desirably occur prior to implantation of a prosthetic heart valve into the mitral valve 408, which may be calcified.

FIG. 20B illustrates a perspective view of a compression system 400 that may be utilized to compress the interventricular septum 402. The compression system 400 may include a first compressive body 410 and a second compressive body 412. The first compressive body 410 may be configured to be positioned on a first side of the interventricular septum 402 proximate the LVOT 350. The second compressive body 412 may be configured to be positioned on a second side of the interventricular septum 402 or on a free wall of a right ventricle of the heart.

The first compressive body 410 and the second compressive body 412 may each comprise a disk that may be configured to apply a force to a portion of the heart. The first compressive body 410 and second compressive body 412 may each be rigid and configured to apply a compressive force. In examples, a portion of the first compressive body 410 or the second compressive body 412 may be compliant and configured to provide a cushion to the portion of the heart being compressed. In examples, one or more of the first compressive body 410 or the second compressive body 412 may be compressed or linearized to pass through an opening that a tether 414 passes through. Such a configuration may allow for one or more of the first compressive body 410 or the second compressive body 412 to be positioned on opposite sides of the interventricular septum 402. Other configurations may be utilized in examples.

In examples, a tether 414 may be configured to decrease a distance between the first compressive body and the second compressive body for improving flow through the LVOT 350. The tether 414 may compress the first compressive body 410 and the second compressive body 412 together. The tether 414 may be configured to compress the bodies 410, 412 together to increase a size of the LVOT 350 in examples.

The tether 414 may comprise a cord, a wire, a braid, or another form of tether that may couple the first compressive body 410 to the second compressive body 412.

Referring to FIG. 20C, in examples, the first compressive body 410 may be deployed to a side of the interventricular septum 402 within the left ventricle 404 and the second compressive body 412 may be deployed to an opposite side of the interventricular septum 402 within the right ventricle. The tether 414 may be tensioned to compress the interventricular septum 402 and reduce a thickness 406 of the interventricular septum 402. The thickness 406 may be reduced proximate the LVOT 350, to reduce the possibility of an obstruction of the LVOT 350 upon deployment of a prosthetic heart valve.

FIG. 20D, for example, illustrates a prosthetic valve 416 deployed to a mitral valve. The use of the compression system 400 may reduce the possibility of obstruction of the LVOT 350 upon deployment of the prosthetic valve 416.

FIG. 21 illustrates a variation in which the second compressive body 412 may be deployed to a free wall of a right ventricle 418. The tether 420 may span the right ventricle 418 and may pull the interventricular septum 402 towards the right ventricle 418 to increase a size of the LVOT 350. The tether 420 may apply the force to the interventricular septum 402 to move the interventricular septum 402 into the right ventricle 418.

Variations in the compression system 400 may be utilized in examples.

Features of the examples of FIGS. 20A-21 may be utilized solely or in combination with any other example disclosed herein.

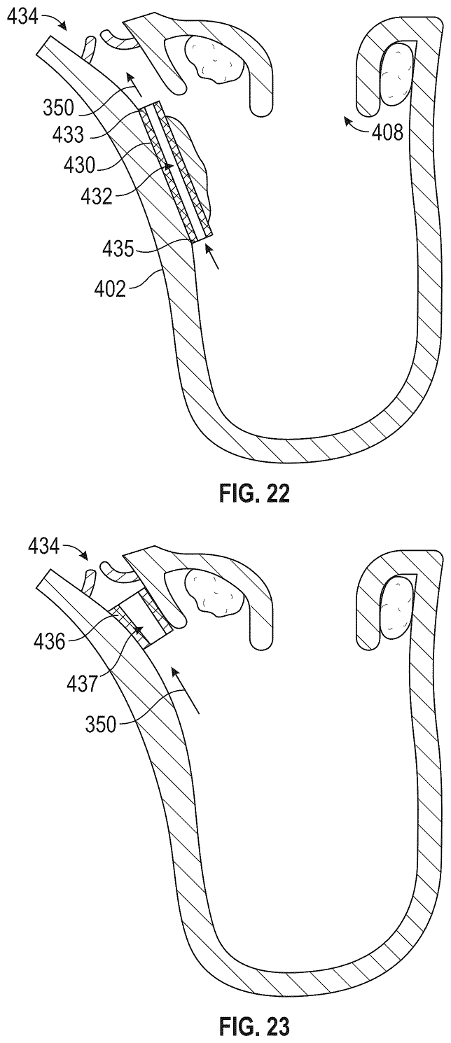

FIG. 22 illustrates use of a stent 430 deployed into the heart proximate the LVOT 350. The stent 430 may include a flow channel 432 for fluid to pass through the LVOT 350.

As shown in FIG. 22, the stent 430 may be passed through the tissue of the heart, for example the tissue of an interior ventricular wall (e.g., the interventricular septum 402) to form a tunnel within the interior ventricular wall. The stent 430 may create a tunnel to the aortic valve 434. A first portion 433 of the stent 430 may be configured to form a first opening of the flow channel 432 proximate the aortic valve 434, and a second portion 435 of the stent 430 may be configured to form a second opening opposite the first opening and at a surface of the interior ventricular wall. The stent 430 may reduce the possibility of obstruction of the LVOT 350 during implantation of a prosthetic valve to the mitral valve 408 or other possible events that may produce an obstruction of the LVOT 350.

Features of the examples of FIG. 22 may be utilized solely or in combination with any other example disclosed herein.

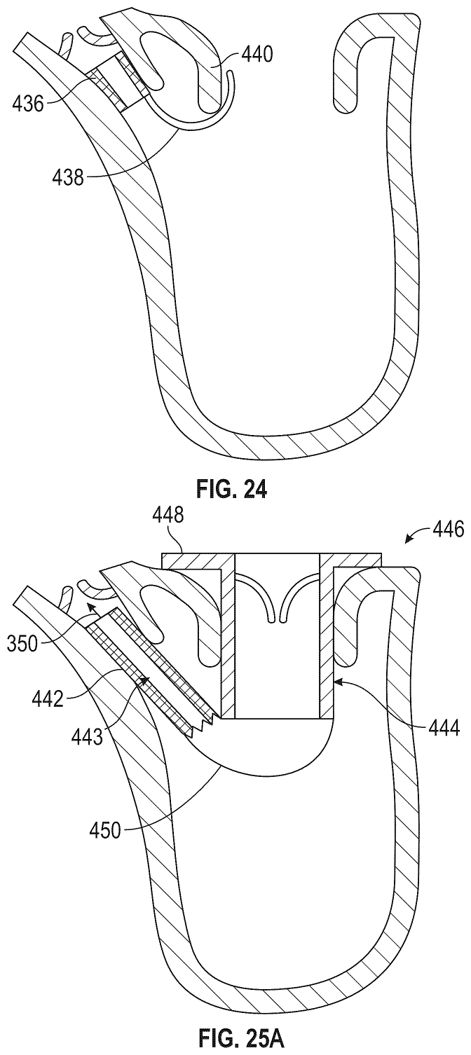

In examples, one or more stents may be alternatively positioned. Referring to FIG. 23, for example, a stent 436 may be positioned proximate the aortic valve 434 and within the LVOT 350. The stent 430 may include a flow channel 437 for fluid to pass through the LVOT 350. Referring to FIG. 24, in examples the stent 436 may include an anchor 438 extending from the stent 436 and configured to anchor to a leaflet 440. The anchor 438 may be configured to hook over the leaflet 440. The leaflet 440 may comprise an anterior leaflet of the mitral valve in examples. The anchor 438 may anchor to the leaflet 440 if the leaflet 440 lacks calcification, or may otherwise anchor to the leaflet 440. A prosthetic heart valve may be deployed to the native mitral valve to provide leaflets for opening and closing at the mitral valve.

Features of the examples of FIGS. 23-24 may be utilized solely or in combination with any other example disclosed herein.

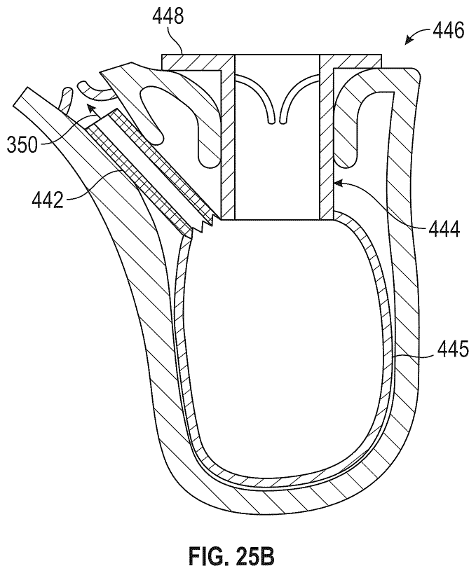

FIG. 25A illustrates an example of a stent 442 that may be positioned within the LVOT 350. The stent 442 may be configured to be deployed into the heart proximate the LVOT 350 and may include a flow channel 443 for fluid to pass through the LVOT 350. The stent 442 may be coupled to a prosthetic heart valve 444 that may be implanted to the mitral valve 446. In examples, the stent 442 may be utilized to anchor the prosthetic heart valve 444. For example, the prosthetic heart valve 444 may include an atrial anchor 448 and the stent 442 may comprise an anchor resisting proximal or atrial movement of the prosthetic heart valve 444.

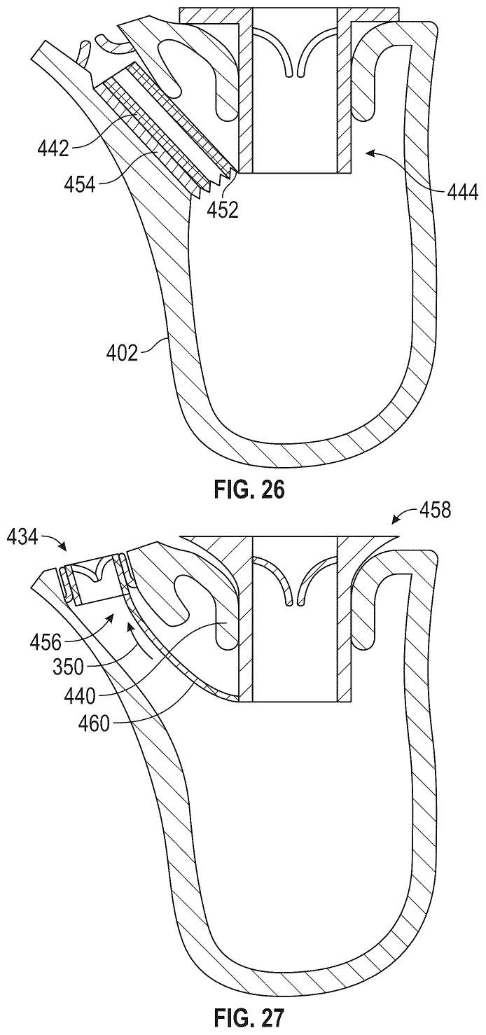

In examples, a curved coupler 450 may be used between the stent 442 and the prosthetic heart valve 444 as shown in FIG. 25A. The curved coupler 450 may arc to the stent 442 within the left ventricle. In examples, and referring to FIG. 25B, a coupler 445 may comprise a bladder that may be positioned within the ventricle. The bladder may be configured to fill with blood, with the motion of the ventricle filling the bladder and ejecting blood from the bladder. The bladder may substantially fill the ventricle in examples. The coupler 445 may couple the stent 442 to the prosthetic heart valve 444 as shown in FIG. 25B. Referring to FIG. 26, in examples, a coupler 452 may couple the stent 442 to an adjacent prosthetic heart valve 444. As shown in FIG. 26, the stent 442 may be coupled to a bumper 454 positioned adjacent to the stent 442 and that may be configured to provide a spacing of the stent from the wall of the interventricular septum 402.

In examples, a prosthetic heart valve 456 may be configured to be implanted in the aortic valve 434 and may be coupled to a prosthetic heart valve 458 configured to be implanted in the mitral valve. FIG. 27 illustrates an exemplary configuration. The aortic prosthetic heart valve 456 may be configured to have one or more prosthetic valve leaflets coupled to a frame and the mitral prosthetic heart valve 458 may include one or more prosthetic valve leaflets coupled to a frame. Other configurations of prosthetic heart valves may be utilized as desired.

A tether 460 may extend from the aortic prosthetic heart valve 456 to the mitral prosthetic heart valve 458. The tether 460 may be configured to impede a mitral valve leaflet 440 from protruding undesirably into the LVOT 350. For example, the tether 460 may extend over the mitral valve leaflet 440 and hook the mitral valve leaflet 440. The aortic prosthetic heart valve 456 may further serve as an anchor for the mitral prosthetic heart valve 458 to impede proximal or atrial movement of the prosthetic heart valve 458.

Features of the examples of FIGS. 25A-27 may be utilized solely or in combination with any other example disclosed herein.

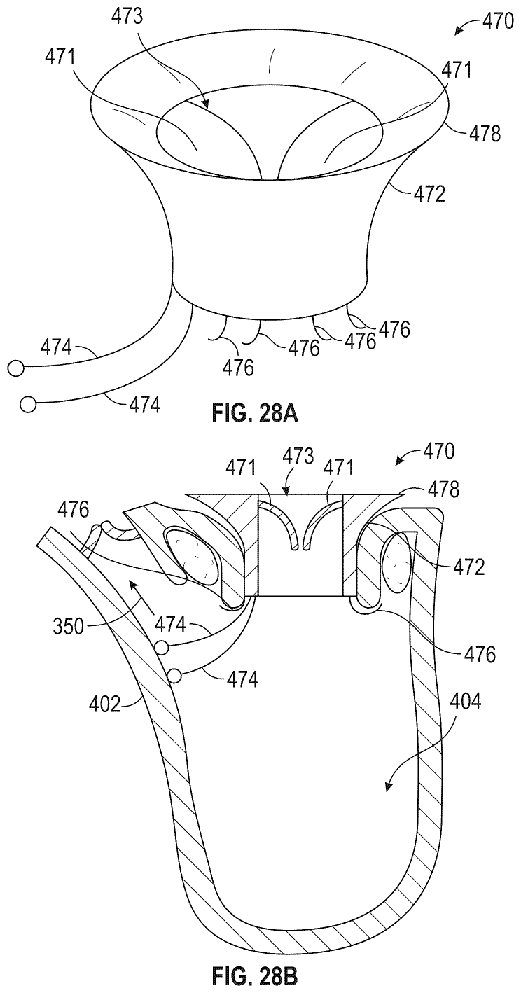

FIGS. 28A-28B illustrates an example of a prosthetic valve 470 including a valve body or support structure 472 and at least one distal arm 474 coupled to the support structure 472 and configured to apply a force to the interventricular septum 402 between the left ventricle 404 and a right ventricle of the heart. The support structure 472 may support one or more prosthetic valve leaflets 471 configured to be positioned in a flow channel 473.

A distal arm 474 may protrude distally and contact the interventricular septum 402 to push the septum 402 away from the LVOT 350 and increase or maintain a size of the LVOT 350. The distal arm 474 may apply a force to the interventricular septum 402 to increase the size of the LVOT 350. The distal arm 474 may extend from the mitral valve across the LVOT 350.

The prosthetic valve 470 may further include one or more distal anchors 476 that may each have a shorter length than the distal arm 474, and may be configured to hook over native valve leaflets having calcification as shown in FIG. 28B.

In examples, the prosthetic valve 470 may include a proximal anchor comprising a flange 478 configured to extend radially outward from the flow channel 473. The flange 478 may protrude and impede distal or ventricular movement of the prosthetic valve 470. The prosthetic valve 470 may be configured to be deployed to a mitral valve.

The examples of prosthetic valves may be utilized in a mitral valve as disclosed herein, or may be utilized in other deployment locations such as a native tricuspid valve, or other deployment locations unless stated otherwise. Deployment to aortic or pulmonary valves, or other implantation sites may be utilized.

Features of the examples of FIGS. 28A-28B may be utilized solely or in combination with any other example disclosed herein.

Features of examples may be utilized solely, or in combination with other features disclosed herein.

Various modifications of the examples disclosed herein may be provided. Features of examples may be modified, substituted, excluded, or combined across examples as desired. Combinations of features across examples may be provided as desired. Combinations of features may be provided across examples with other features of such examples being excluded if desired.

The implants disclosed herein may include prosthetic heart valves or other forms of implants, such as stents or filters, or diagnostic devices, among others. The implants may be expandable implants configured to move from a compressed or undeployed state to an expanded or deployed state. The implants may be compressible implants configured to be compressed inward to have a reduced outer profile and to move the implant to the compressed or undeployed state.

Various forms of delivery apparatuses may be utilized with the examples disclosed herein. The delivery apparatuses as disclosed herein may be utilized for aortic, mitral, tricuspid, and pulmonary replacement and repair as well. The delivery apparatuses may comprise delivery apparatuses for delivery of other forms of implants, such as stents or filters, or diagnostic devices, among others.

The implants and the systems disclosed herein may be used in transcatheter mitral or tricuspid implantation, as well as aortic valve implantation (TAVI) or replacement of other native heart valves (e.g., pulmonary valves). The delivery apparatuses and the systems disclosed herein may be utilized for transarterial access, including transfemoral access, to a patient's heart. The delivery apparatuses and systems may be utilized in transcatheter percutaneous procedures, including transarterial procedures, which may be transfemoral or transjugular. Transapical procedures, among others, may also be utilized. Other procedures may be utilized as desired.

In addition, the methods herein are not limited to the methods specifically described, and may include methods of utilizing the systems and apparatuses disclosed herein. The steps of the methods may be modified, excluded, or added to, with systems, apparatuses, and methods disclosed herein. The examples disclosed herein may comprise systems for implantation within a human body in examples.

For purposes of this description, certain aspects, advantages, and novel features of the examples of this disclosure are described herein. The disclosed methods, apparatuses, and systems should not be construed as limiting in any way. Instead, the present disclosure is directed toward all novel and nonobvious features and aspects of the various disclosed examples, along and in various combinations and sub-combinations with one another. The methods, apparatuses, and systems are not limited to any specific aspect or feature or combination thereof, nor do the disclosed examples require that any one or more specific advantages be present or problems be solved. Features, elements, or combinations of one example can be combined into other examples herein.

Example 1: A prosthetic valve configured to be deployed to a native valve, the prosthetic valve comprising: one or more prosthetic valve leaflets configured to be positioned in a flow channel; and a valve body configured to support the one or more prosthetic valve leaflets, the valve body including: a proximal anchor comprising a first flange configured to extend radially outward from the flow channel, and a distal anchor comprising a second flange configured to extend radially outward from the flow channel.

Example 2: The prosthetic valve of any example herein, in particular, example 1, wherein the first flange comprises a first disk and the second flange comprises a second disk.

Example 3: The prosthetic valve of any example herein, in particular, example 2, wherein the valve body is configured to expand radially outward to an expanded configuration, the first disk including a first end portion and a second end portion and a protruding portion configured to protrude radially outward from the first end portion and the second end portion, and the first end portion and the second end portion are configured to move towards each other when the valve body expands radially outward to the expanded configuration.

Example 4: The prosthetic valve of any example herein, in particular, example 3, wherein the protruding portion comprises a loop of material.

Example 5: The prosthetic valve of any example herein, in particular, examples 1-4, wherein the first flange comprises a mesh and the second flange comprises a mesh.

Example 6: The prosthetic valve of any example herein, in particular, examples 1-5, wherein the first flange and the second flange each comprise an inflatable body.

Example 7: The prosthetic valve of any example herein, in particular, examples 1-6, wherein the first flange and the second flange each include a compressible cloth or a compressible foam.

Example 8: The prosthetic valve of any example herein, in particular, examples 1-7, wherein the valve body includes a frame, and the first flange and the second flange each comprise ridges of the frame.

Example 9: The prosthetic valve of any example herein, in particular, examples 1-8, wherein the valve body includes a frame, and the first flange and the second flange each comprise cut out portions of the frame having ends that are configured to protrude radially outward from the frame and angle towards each other.

Example 10: The prosthetic valve of any example herein, in particular, examples 1-9, wherein the first flange and the second flange each comprise barbs.

Example 11: The prosthetic valve of any example herein, in particular, examples 1-10, wherein the valve body is configured to have a cylindrical configuration and an expanded configuration, the first flange and the second flange each being retracted in the cylindrical configuration and configured to move radially outward in the expanded configuration.

Example 12: The prosthetic valve of any example herein, in particular, examples 1-11, wherein the valve body is configured to dock with an insert retaining the one or more prosthetic valve leaflets.

Example 13: The prosthetic valve of any example herein, in particular, examples 1-12, wherein the first flange is configured to be positioned on a proximal side of the native valve, and the second flange is configured to be positioned on a distal side of the native valve.

Example 14: The prosthetic valve of any example herein, in particular, examples 1-13, wherein the first flange is configured to be positioned on an atrial side of a native mitral valve or a native tricuspid valve, and the second flange is configured to be positioned on a ventricular side of the native mitral valve or the native tricuspid valve.

Example 15: The prosthetic valve of any example herein, in particular, examples 1-14, wherein the first flange is configured to anchor to calcification of the native valve and the second flange is configured to anchor to calcification of the native valve.

Example 16: A method comprising: deploying a prosthetic valve to a native valve, the prosthetic valve comprising: one or more prosthetic valve leaflets configured to be positioned in a flow channel, and a valve body configured to support the one or more prosthetic valve leaflets, the valve body including: a proximal anchor comprising a first flange configured to extend radially outward from the flow channel, and a distal anchor comprising a second flange configured to extend radially outward from the flow channel.

Example 17: The method of any example herein, in particular, example 16, wherein the first flange comprises a first disk and the second flange comprises a second disk.

Example 18: The method of any example herein, in particular, example 16 or example 17, wherein the first flange comprises a mesh and the second flange comprises a mesh.

Example 19: The method of any example herein, in particular, examples 16-18, wherein the first flange and the second flange each comprise an inflatable body.

Example 20: The method of any example herein, in particular, examples 16-19, wherein the first flange and the second flange each include a compressible cloth or a compressible foam.

Example 21: The method of any example herein, in particular, examples 16-20, wherein the valve body includes a frame, and the first flange and the second flange each comprise ridges of the frame.

Example 22: The method of any example herein, in particular, examples 16-21, wherein the valve body includes a frame, and the first flange and the second flange each comprise cut out portions of the frame having ends that are configured to protrude radially outward from the frame and angle towards each other.

Example 23: The method of any example herein, in particular, examples 16-22, wherein the first flange is configured to be positioned on a proximal side of the native valve, and the second flange is configured to be positioned on a distal side of the native valve.

Example 24: The method of any example herein, in particular, examples 16-23, wherein the first flange is configured to be positioned on an atrial side of a native mitral valve or a native tricuspid valve, and the second flange is configured to be positioned on a ventricular side of the native mitral valve or the native tricuspid valve.

Example 25: The method of any example herein, in particular, examples 16-24, wherein the first flange is configured to anchor to calcification of the native valve and the second flange is configured to anchor to calcification of the native valve.

Example 26: A prosthetic valve configured to be deployed to a native valve, the prosthetic valve comprising: one or more prosthetic valve leaflets configured to be positioned in a flow channel; and a valve body configured to support the one or more prosthetic valve leaflets, the valve body including an atrial anchor forming a ring about the flow channel.

Example 27: The prosthetic valve of any example herein, in particular, example 26, wherein the ring includes an upper surface configured to face in an atrial direction and a lower surface configured to face opposite the upper surface and in a ventricular direction, and the prosthetic valve further comprises a compliant body positioned on the lower surface.

Example 28: The prosthetic valve of any example herein, in particular, example 27, wherein the compliant body comprises one or more of an inflatable body or a foam.

Example 29: The prosthetic valve of any example herein, in particular, examples 26-28, wherein the ring extends radially outward from the flow channel.

Example 30: The prosthetic valve of any example herein, in particular, examples 26-29, wherein the ring comprises a disk.

Example 31: The prosthetic valve of any example herein, in particular, examples 26-30, further comprising one or more penetrating bodies configured to pass through the ring to anchor the valve body to the native valve.

Example 32: The prosthetic valve of any example herein, in particular, examples 26-31, wherein the ring has a tubular shape and the one or more prosthetic valve leaflets are coupled to the ring and are configured to overlay one or more native valve leaflets of the native valve.

Example 33: The prosthetic valve of any example herein, in particular, examples 26-32, wherein the atrial anchor comprises a gasket.

Example 34: The prosthetic valve of any example herein, in particular, example 33, wherein the gasket is conformable and configured to conform to a shape of calcification of the native valve.

Example 35: The prosthetic valve of any example herein, in particular, examples 26-34, wherein the atrial anchor is configured to anchor to calcification of the native valve.

Example 36: A method comprising: deploying a prosthetic valve to a native valve, the prosthetic valve comprising: one or more prosthetic valve leaflets configured to be positioned in a flow channel, and a valve body configured to support the one or more prosthetic valve leaflets, the valve body including an atrial anchor forming a ring about the flow channel.

Example 37: The method of any example herein, in particular, example 36, wherein the ring includes an upper surface configured to face in an atrial direction and a lower surface configured to face opposite the upper surface and in a ventricular direction, and the prosthetic valve further comprises a compliant body positioned on the lower surface.

Example 38: The method of any example herein, in particular, example 37, wherein the compliant body comprises one or more of an inflatable body or a foam.

Example 39: The method of any example herein, in particular, examples 36-38, wherein the ring extends radially outward from the flow channel.

Example 40: The method of any example herein, in particular, examples 36-39, wherein the ring comprises a disk.

Example 41: The method of any example herein, in particular, examples 36-40, further comprising one or more penetrating bodies configured to pass through the ring to anchor the valve body to the native valve.

Example 42: The method of any example herein, in particular, examples 36-41, wherein the ring has a tubular shape and the one or more prosthetic valve leaflets are coupled to the ring and are configured to overlay one or more native valve leaflets of the native valve.

Example 43: The method of any example herein, in particular, examples 36-42, wherein the atrial anchor comprises a gasket.

Example 44: The method of any example herein, in particular, example 43, wherein the gasket is conformable and configured to conform to a shape of calcification of the native valve.

Example 45: The method of any example herein, in particular, examples 36-44, wherein the atrial anchor is configured to anchor to calcification of the native valve.

Example 46: A prosthetic valve system configured to be deployed to a native valve, the prosthetic valve system comprising: one or more prosthetic valve leaflets configured to be positioned in a flow channel; a valve body supporting the one or more prosthetic valve leaflets and surrounding the flow channel; and a dock comprising an elongate body having a first end portion and a second end portion and configured to form a crescent shape, the first end portion including a first penetrating body configured to anchor to heart tissue and the second end portion including a second penetrating body configured to anchor to heart tissue, the dock configured to dock with the valve body.

Example 47: The prosthetic valve of any example herein, in particular, example 46, wherein the first penetrating body comprises a screw.

Example 48: The prosthetic valve of any example herein, in particular, example 46 or example 47, wherein the valve body includes an outer surface comprising a sealing surface.

Example 49: The prosthetic valve of any example herein, in particular, examples 46-48, wherein the dock is configured to extend around a radially outward surface of a leaflet of the native valve and position the leaflet between the dock and an outer surface of the valve body.

Example 50: The prosthetic valve of any example herein, in particular, examples 46-49, wherein the dock is configured to be deployed to a native mitral valve or a native tricuspid valve.

Example 51: A method comprising: deploying a dock proximate a native valve, the dock comprising an elongate body having a first end portion and a second end portion and configured to form a crescent shape, the first end portion including a first penetrating body configured to anchor to heart tissue and the second end portion including a second penetrating body configured to anchor to heart tissue; and deploying a valve body supporting one or more prosthetic valve leaflets to the dock.

Example 52: The method of any example herein, in particular, example 51, wherein the first penetrating body comprises a screw.

Example 53: The method of any example herein, in particular, example 51 or example 52, wherein the valve body includes an outer surface comprising a sealing surface.

Example 54: The method of any example herein, in particular, examples 51-53, wherein the dock extends around a radially outward surface of a leaflet of the native valve and the leaflet is positioned between the dock and an outer surface of the valve body.

Example 55: The method of any example herein, in particular, examples 51-54, further comprising deploying the dock to a native mitral valve or a native tricuspid valve.

Example 56: A prosthetic valve configured to be deployed to a native valve, the prosthetic valve comprising: one or more prosthetic valve leaflets configured to be positioned in a flow channel; and a valve body configured to support the one or more prosthetic valve leaflets, the valve body including an outer perimeter including a first portion forming at least a quarter of the outer perimeter and a second portion forming a remainder of the outer perimeter, and the valve body includes one or more distal anchors protruding from the second portion and lacks distal anchors protruding from the first portion.

Example 57: The prosthetic valve of any example herein, in particular, example 56, wherein each of the one or more distal anchors is configured to hook over a distal tip of a leaflet of the native valve.

Example 58: The prosthetic valve of any example herein, in particular, example 56 or example 57, wherein the first portion comprises a sealing surface configured to form a seal with the native valve.

Example 59: The prosthetic valve of any example herein, in particular, examples 56-58, wherein the first portion forms at least a half of the outer perimeter.

Example 60: The prosthetic valve of any example herein, in particular, examples 56-59, wherein the prosthetic valve is configured to be deployed to a mitral valve, and the first portion comprises a posterior portion of the valve body.

Example 61: A method comprising: deploying a prosthetic valve to a native valve, the prosthetic valve comprising: one or more prosthetic valve leaflets configured to be positioned in a flow channel, and a valve body configured to support the one or more prosthetic valve leaflets, the valve body including an outer perimeter including a first portion forming at least a quarter of the outer perimeter and a second portion forming a remainder of the outer perimeter, and the valve body includes one or more distal anchors protruding from the second portion and lacks distal anchors protruding from the first portion.

Example 62: The method of any example herein, in particular, example 61, wherein each of the one or more distal anchors is configured to hook over a distal tip of a leaflet of the native valve.

Example 63: The method of any example herein, in particular, example 61 or example 62, wherein the first portion comprises a sealing surface configured to form a seal with the native valve.

Example 64: The method of any example herein, in particular, examples 61-63, wherein the first portion forms at least a half of the outer perimeter.

Example 65: The method of any example herein, in particular, examples 61-64, further comprising deploying the prosthetic valve to a mitral valve, and the first portion comprises a posterior portion of the valve body.

Example 66: A prosthetic valve configured to be deployed to a native valve, the prosthetic valve comprising: one or more prosthetic valve leaflets configured to be positioned in a flow channel; and a valve body configured to support the one or more prosthetic valve leaflets and including a sealing body having a first portion and a second portion, the first portion impeding fluid flow at a height of the valve body and the second portion being at the height of the first portion and being at a circumferentially offset position from the position of the first portion, the second portion being recessed to allow for fluid flow through at the circumferentially offset position.

Example 67: The prosthetic valve of any example herein, in particular, example 66, wherein the valve body includes a frame including a plurality of openings bound by a plurality of struts, and the sealing body at the circumferentially offset position does not cover at least one of the plurality of openings to allow for fluid flow through.

Example 68: The prosthetic valve of any example herein, in particular, example 66 or example 67, wherein the valve body includes a frame that is recessed at the circumferentially offset position.

Example 69: The prosthetic valve of any example herein, in particular, examples 66-68, wherein the sealing body comprises a sealing skirt.

Example 70: The prosthetic valve of any example herein, in particular, examples 66-69, wherein the prosthetic valve is configured to be deployed to a mitral valve, and the circumferentially offset position comprises an anterior portion of the valve body.

Example 71: A method comprising: deploying a prosthetic valve to a native valve, the prosthetic valve comprising: one or more prosthetic valve leaflets configured to be positioned in a flow channel, and a valve body configured to support the one or more prosthetic valve leaflets and including a sealing body having a first portion and a second portion, the first portion impeding fluid flow at a height of the valve body and the second portion being at the height of the first portion and being at a circumferentially offset position from the position of the first portion, the second portion being recessed to allow for fluid flow through at the circumferentially offset position.

Example 72: The method of any example herein, in particular, example 71, wherein the valve body includes a frame including a plurality of openings bound by a plurality of struts, and the sealing body at the circumferentially offset position does not cover at least one of the plurality of openings to allow for fluid flow through.

Example 73: The method of any example herein, in particular, example 71 or example 72, wherein the valve body includes a frame that is recessed at the circumferentially offset position.

Example 74: The method of any example herein, in particular, examples 71-73, wherein the sealing body comprises a sealing skirt.

Example 75: The method of any example herein, in particular, examples 71-74, further comprising deploying the prosthetic valve to a mitral valve, and the circumferentially offset position comprises an anterior portion of the valve body.

Example 76: A prosthetic valve configured to be deployed to a native valve, the prosthetic valve comprising: one or more prosthetic valve leaflets configured to be positioned in a flow channel; a valve body configured to support the one or more prosthetic valve leaflets; and at least one distal anchor coupled to the valve body, the at least one distal anchor including a hinge forming a loop and a straight portion configured to extend radially outward from the loop.

Example 77: The prosthetic valve of any example herein, in particular, example 76, wherein the straight portion is configured to rotate about the hinge in a proximal direction.

Example 78: The prosthetic valve of any example herein, in particular, example 76 or example 77, wherein the straight portion is positioned between the loop and a tip of the at least one distal anchor, the tip being angled relative to the straight portion.

Example 79: The prosthetic valve of any example herein, in particular, examples 76-78, wherein the loop protrudes radially inward.

Example 80: The prosthetic valve of any example herein, in particular, examples 76-79, wherein the at least one distal anchor comprises an arm, and the loop is positioned between the straight portion and a coupling portion configured to couple the loop to the valve body.

Example 81: A method comprising: deploying a prosthetic valve to a native valve, the prosthetic valve comprising: one or more prosthetic valve leaflets configured to be positioned in a flow channel, a valve body configured to support the one or more prosthetic valve leaflets, and at least one distal anchor coupled to the valve body, the at least one distal anchor including a hinge forming a loop and a straight portion configured to extend radially outward from the loop.

Example 82: The method of any example herein, in particular, example 81, wherein the straight portion is configured to rotate about the hinge in a proximal direction.

Example 83: The method of any example herein, in particular, example 81 or example 82, wherein the straight portion is positioned between the loop and a tip of the at least one distal anchor, the tip being angled relative to the straight portion.

Example 84: The method of any example herein, in particular, examples 81-83, wherein the loop protrudes radially inward.

Example 85: The method of any example herein, in particular, examples 81-84, wherein the at least one distal anchor comprises an arm, and the loop is positioned between the straight portion and a coupling portion configured to couple the loop to the valve body.

Example 86: A compression system for a heart comprising: a first compressive body configured to be positioned on a first side of an interventricular septum of the heart proximate a left ventricular outflow tract; a second compressive body configured to be positioned on a second side of the interventricular septum or on a free wall of a right ventricle of the heart; and a tether configured to compress the first compressive body and the second compressive body together to increase a size of the left ventricular outflow tract.

Example 87: The compression system of any example herein, in particular, example 86, wherein the first compressive body and the second compressive body each comprise a disk.

Example 88: The compression system of any example herein, in particular, example 86 or example 87, wherein the first compressive body is configured to apply a force to the interventricular septum to move the interventricular septum into the right ventricle.

Example 89: The compression system of any example herein, in particular, examples 86-88, wherein the first compressive body is configured to apply a force to the interventricular septum to reduce a thickness of the interventricular septum.

Example 90: The compression system of any example herein, in particular, examples 86-89, wherein the second compressive body is configured to be compressed and passed through an opening in the interventricular septum that the tether passes through.

Example 91: A method comprising: deploying a first compressive body on a first side of an interventricular septum of a heart proximate a left ventricular outflow tract; deploying a second compressive body on a second side of the interventricular septum or on a free wall of a right ventricle of the heart; and tensioning a tether extending between the first compressive body and the second compressive body to compress the first compressive body and the second compressive body together to increase a size of the left ventricular outflow tract.

Example 92: The method of any example herein, in particular, example 91, wherein the first compressive body and the second compressive body each comprise a disk.

Example 93: The method of any example herein, in particular, example 91 or example 92, wherein the first compressive body applies a force to the interventricular septum to move the interventricular septum into the right ventricle.

Example 94: The method of any example herein, in particular, examples 91-93, wherein the first compressive body applies a force to the interventricular septum to reduce a thickness of the interventricular septum.

Example 95: The method of any example herein, in particular, examples 91-94, wherein the second compressive body is passed through an opening in the interventricular septum that the tether passes through.

Example 96: A system for a heart, the system comprising: a stent configured to be deployed into the heart proximate a left ventricular outflow tract of the heart and including a flow channel for fluid to pass through the left ventricular outflow tract.

Example 97: The system of any example herein, in particular, example 96, further comprising an anchor extending from the stent and configured to anchor to a leaflet of a mitral valve of the heart.

Example 98: The system of any example herein, in particular, example 96 or example 97, further comprising a prosthetic valve coupled to the stent and configured to be implanted to a mitral valve of the heart.

Example 99: The system of any example herein, in particular, examples 96-98, wherein the stent is configured to be passed through tissue of an interior ventricular wall to form a tunnel within the interior ventricular wall.

Example 100: The system of any example herein, in particular, example 99, wherein a first end portion of the stent is configured to form a first opening of the flow channel proximate an aortic valve of the heart, and a second portion of the stent is configured to form a second opening opposite the first opening and at a surface of the interior ventricular wall.

Example 101: A method comprising: deploying a stent proximate a left ventricular outflow tract of a heart, the stent including a flow channel for fluid to pass through the left ventricular outflow tract.

Example 102: The method of any example herein, in particular, example 101, wherein an anchor extends from the stent and anchors to a leaflet of a mitral valve of the heart.

Example 103: The method of any example herein, in particular, example 101 or example 102, wherein a prosthetic valve is coupled to the stent and is implanted to a mitral valve of the heart.

Example 104: The method of any example herein, in particular, examples 101-103, wherein the stent passes through tissue of an interior ventricular wall to form a tunnel within the interior ventricular wall.

Example 105: The method of any example herein, in particular, example 104, wherein a first end portion of the stent forms a first opening of the flow channel proximate an aortic valve of the heart, and a second portion of the stent forms a second opening opposite the first opening and at a surface of the interior ventricular wall.

Example 106: A system for a heart, the system comprising: a first prosthetic heart valve configured to be implanted in an aortic valve of the heart; and a second prosthetic heart valve coupled to the first prosthetic heart valve and configured to be implanted in a mitral valve of the heart.

Example 107: The system of any example herein, in particular, example 106, wherein a tether couples the first prosthetic heart valve to the second prosthetic heart valve and is configured to extend over a native valve leaflet of the mitral valve.

Example 108: The system of any example herein, in particular, example 107, wherein the tether is configured to hook the native valve leaflet.

Example 109: The system of any example herein, in particular, examples 106-108, wherein the first prosthetic heart valve includes one or more prosthetic valve leaflets coupled to a frame.

Example 110: The system of any example herein, in particular, examples 106-109, wherein the second prosthetic heart valve includes one or more prosthetic valve leaflets coupled to a frame.

Example 111: A method comprising: implanting a first prosthetic heart valve in an aortic valve of a heart; and implanting a second prosthetic heart valve to a mitral valve of the heart, the second prosthetic heart valve being coupled to the first prosthetic heart valve.

Example 112: The method of any example herein, in particular, example 111, wherein a tether couples the first prosthetic heart valve to the second prosthetic heart valve and extends over a native valve leaflet of the mitral valve.

Example 113: The method of any example herein, in particular, example 112, wherein the tether hooks the native valve leaflet.

Example 114: The method of any example herein, in particular, examples 111-113, wherein the first prosthetic heart valve includes one or more prosthetic valve leaflets coupled to a frame.

Example 115: The method of any example herein, in particular, examples 111-114, wherein the second prosthetic heart valve includes one or more prosthetic valve leaflets coupled to a frame.

Example 116: A prosthetic valve configured to be deployed to a native valve of a heart, the prosthetic valve comprising: one or more prosthetic valve leaflets configured to be positioned in a flow channel; a valve body configured to support the one or more prosthetic valve leaflets; and at least one distal arm coupled to the valve body and configured to apply a force to an interventricular septum between a left ventricle and a right ventricle of the heart.

Example 117: The prosthetic valve of any example herein, in particular, example 116, wherein the at least one distal arm is configured to apply the force to the interventricular septum to increase a size of a left ventricular outflow tract of the heart.

Example 118: The prosthetic valve of any example herein, in particular, example 116 or example 117, wherein the at least one distal arm is configured to extend from a mitral valve of the heart across a left ventricular outflow tract of the heart.

Example 119: The prosthetic valve of any example herein, in particular, examples 116-118, further comprising a plurality of distal anchors each having a shorter length than the at least one distal arm.

Example 120: The prosthetic valve of any example herein, in particular, examples 116-119, wherein the valve body includes a proximal anchor comprising a flange configured to extend radially outward from the flow channel.

Example 121: A method comprising: deploying a prosthetic valve to a native valve, the prosthetic valve comprising: one or more prosthetic valve leaflets configured to be positioned in a flow channel, a valve body configured to support the one or more prosthetic valve leaflets, and at least one distal arm coupled to the valve body and configured to apply a force to an interventricular septum between a left ventricle and a right ventricle of a heart.

Example 122: The method of any example herein, in particular, example 121, wherein the at least one distal arm applies the force to the interventricular septum to increase a size of a left ventricular outflow tract of the heart.

Example 123: The method of any example herein, in particular, example 121 or example 122, wherein the at least one distal arm extends from a mitral valve of the heart across a left ventricular outflow tract of the heart.

Example 124: The method of any example herein, in particular, examples 121-123, further comprising a plurality of distal anchors each having a shorter length than the at least one distal arm.

Example 125: The method of any example herein, in particular, examples 121-124, wherein the valve body includes a proximal anchor comprising a flange configured to extend radially outward from the flow channel.

Any of the features of any of the examples, including but not limited to any of the first through 125 examples referred to above, is applicable to all other aspects and examples identified herein, including but not limited to any examples of any of the first through 125 examples referred to above. Moreover, any of the features of an example of the various examples, including but not limited to any examples of any of the first through 125 examples referred to above, is independently combinable, partly or wholly with other examples described herein in any way, e.g., one, two, or three or more examples may be combinable in whole or in part. Further, any of the features of the various examples, including but not limited to any examples of any of the first through 125 examples referred to above, may be made optional to other examples. Any example of a method can be performed by a system or apparatus of another example, and any aspect or example of a system or apparatus can be configured to perform a method of another aspect or example, including but not limited to any examples of any of the first through 125 examples referred to above.

In closing, it is to be understood that although aspects of the present specification are highlighted by referring to specific examples, one skilled in the art will readily appreciate that these disclosed examples are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular examples only, and is not intended to limit the scope of systems, apparatuses, and methods as disclosed herein, which is defined solely by the claims. Accordingly, the systems, apparatuses, and methods are not limited to that precisely as shown and described.

Certain examples of systems, apparatuses, and methods are described herein, including the best mode known to the inventors for carrying out the same. Of course, variations on these described examples will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described examples in all possible variations thereof is encompassed by the systems, apparatuses, and methods unless otherwise indicated herein or otherwise clearly contradicted by context.

Groupings of alternative examples, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A prosthetic valve for replacing a calcified native valve, the prosthetic valve comprising:
an inner support stent having an inlet end portion and an outlet end portion, the inner support stent made from a shape memory material;
a valve portion positioned within a passageway of the inner support stent, wherein the valve portion comprises a plurality of leaflets made from pericardium, wherein the valve portion permits flow of blood through the passageway in one direction for replacing the function of the calcified native valve; and
an outer conformable structure surrounding the inner support stent and including a first flange and a second flange each made of a mesh and adapted for anchoring to the calcified native valve, wherein the first flange is adapted to be positioned on a proximal side of the calcified native valve, and the second flange is adapted to be positioned on a distal side of the calcified native valve, and
wherein the outer conformable structure is adapted to conform to calcification on the calcified native valve.

2. The prosthetic valve of claim 1, wherein the first flange comprises a first disk and the second flange comprises a second disk.

3. The prosthetic valve of claim 2, wherein the outer conformable structure is adapted to expand radially outward to an expanded configuration, the first disk including a first end portion and a second end portion and a protruding portion for protruding radially outward from the first end portion and the second end portion, and the first end portion and the second end portion are adapted to move towards each other when the outer conformable structure expands radially outward to the expanded configuration.

4. The prosthetic valve of claim 3, wherein the first end portion and the second end portion are adapted to move towards each other in an axial direction when the outer conformable structure expands radially outward to the expanded configuration.

5. The prosthetic valve of claim 3, wherein the protruding portion comprises a loop of material.

6. The prosthetic valve of claim 1, wherein the inner support stent has a generally cylindrical configuration and an expanded configuration, the first flange and the second flange each being retracted in the cylindrical configuration and adapted to move radially outward in the expanded configuration.

7. The prosthetic valve of claim 1, wherein the first flange is adapted to be positioned on an atrial side of the calcified native valve, and the second flange is adapted to be positioned on a ventricular side of the calcified native valve.

8. The prosthetic valve of claim 1, wherein the valve portion comprises an insert adapted to dock with the inner support stent.

9. A prosthetic valve for replacing a calcified native valve, the prosthetic valve comprising:
a support structure for deployment on an upstream side of the calcified native valve and including an atrial anchor forming a ring about a flow channel; and
a plurality of leaflets made from pericardium coupled to the support structure and disposed within a passageway of the support structure for providing one-way flow of blood.

10. The prosthetic valve of claim 9, wherein the ring includes an upper surface for facing in an atrial direction and a lower surface for facing opposite the upper surface and in a ventricular direction, and the prosthetic valve further comprises a compliant body positioned on the lower surface.

11. The prosthetic valve of claim 10, wherein the compliant body comprises one or more of an inflatable body or a foam.

12. The prosthetic valve of claim 9, wherein the ring extends radially outward from the flow channel and comprises a disk.

13. The prosthetic valve of claim 9, wherein the ring has a tubular shape, and the plurality of leaflets are coupled to the ring and are adapted to overlay native valve leaflets of the calcified native valve.

14. The prosthetic valve of claim 13, wherein each of the plurality of leaflets includes a distal end portion having a barb for anchoring to calcification on the downstream side of the native valve leaflets.

15. A system comprising:
a prosthetic valve for implantation within a native valve, the prosthetic valve including:
a support structure having an outer perimeter including a first portion forming at least a quarter of the outer perimeter and a second portion forming a remainder of the outer perimeter, and the support structure includes one or more distal anchors for capturing a native valve leaflet protruding from the second portion and lacks distal anchors protruding from the first portion, and
one or more prosthetic valve leaflets for being positioned in a flow channel of the support structure.

16. The system of claim 15, wherein each of the one or more distal anchors includes a hinge forming a loop and a straight portion configured to extend radially outward from the loop.

17. The system of claim 15, wherein the first portion comprises one or more barbs protruding from the outer perimeter and for anchoring with the native valve, and wherein the prosthetic valve is for deployment to a calcified valve, and the first portion comprises a posterior portion of the support structure.

18. The system of claim 15, wherein the first portion forms at least a half of the outer perimeter.

19. The system of claim 15, further comprising a compression system for a heart, the compression system including:
a first compressive body for being positioned on a first side of an interventricular septum of the heart proximate a left ventricular outflow tract;
a second compressive body for being positioned on a second side of the interventricular septum or on a free wall of a right ventricle of the heart; and
a tether for decreasing a distance between the first compressive body and the second compressive body for improving flow through the left ventricular outflow tract.

20. The system of claim 19, wherein the first compressive body and the second compressive body each comprise a disk, and wherein the second compressive body is adapted to be compressed and passed through an opening in the interventricular septum that the tether passes through.
